(19) United States
(12) Patent Application Publication
(10) Pub. No.: US 2012/0164740 A1
(43) Pub. Date: Jun. 28, 2012

(54) FLUORESCENT CHEMODOSIMETERS FOR MERCURY IONS BASED ON THE OXYMERCURATION OF VINYL ETHERS

(76) Inventors: Kazunori Koide, Pittsburgh, PA (US); Shin Ando, Kumamoto (JP)

(21) Appl. No.: 13/334,865

(22) Filed: Dec. 22, 2011

Related U.S. Application Data
(60) Provisional application No. 61/426,197, filed on Dec. 22, 2010.

(51) Int. Cl. G01N 21/64; C07D 311/86

(52) U.S. Cl. 436/81; 549/388

(57) ABSTRACT

The present invention is a method to produce and to use a fluorogenic chemodosimeter for the detection of mercury ions in a sample at temperature ranges from about 0° C. to 100° C.

### CROSS REFERENCE TO RELATED APPLICATIONS

This non-provisional application claims priority from U.S. Provisional Patent Application Ser. No. 61/426,197, entitled FLUORESCENT CHEMODOSIMETERS FOR MERCURY IONS BASED ON THE OXYMERCURATION OF VINYL ETHERS filed on Dec. 22, 2010, which is incorporated herein by reference.

### STATEMENT REGARDING FEDERALLY SPONSORED RESEARCH OR DEVELOPMENT

The invention was made with government support under grant numbers CHE-0616577 and CHE-0911092 awarded by the US National Science Foundation and grant number R01CA120792 by the National Institutes of Health. The United States Government has certain rights to the invention.

### FIELD OF THE INVENTION

This invention relates to a method to produce and to use a fluorogenic chemodosimeter that reacts with mercury ions through the oxymercuration of a vinyl ether at temperatures ranging from 0° C. to 100° C. where the product of the reaction is green-fluorescent, allowing for the indirect detection of mercury by fluorescence signals.

### BACKGROUND OF THE INVENTION

Mercury is generally quantified by cold vapor atomic absorption spectroscopy or inductively coupled plasma mass spectroscopy. Although these methods are quantitative and powerful, the analyses require large and expensive instruments, highly trained personnel, and tedious maintenance. Mostly because of the size of the instruments, mercury-contaminated samples are generally analyzed off site.

Optical methods are more amenable to the on-site analysis of mercury with fewer resources. Therefore, numerous fluorescent chemosensors and chemodosimeters have been reported in the literature. These methods may facilitate mercury analyses, even if they are only semiquantitative. A vast majority of the chemosensors and chemodosimeters for mercury contain sulfur atom(s) that can tightly coordinate the metal as part of off-on fluorescence switches.

As the protocol of the US EPA indicates, mercury-containing environmental samples are pretreated with harsh oxidants such as Cl—Br and H₂O₂ to transform various forms of organic and sulfur-bound (e.g., cysteine-bound) mercury species to sulfur-free inorganic mercury(II). Therefore, chemodosimeters and chemosensors for mercury in environmental and biological fluid samples must be compatible with oxidants. The past invention from the inventor's laboratory focused on the t-electrophilicity of mercury ions and the development of a chemodosimeter based on the oxymercuration of an alkyne. The past embodiments of the present invention chemodosimeter for mercury ions were resistant to strong oxidants such as H₂O₂ and N-chlorosuccinimide (NCS). This method enabled detection of mercury from fish and dental samples. However, the oxymercuration reaction needed to be heated to 90° C. Moreover, detection of mercury ions below 4 ppb was not achievable. The ability to detect mercury ions below this level is critically important because the limit of mercury concentration in drinking water is 2 ppb in the United States. It is hypothesized that a more electron-rich π bond might be more reactive toward mercury ions, allowing for mercury detection at a lower temperature and at lower mercury concentrations.

### SUMMARY OF THE INVENTION

The present invention is a fluorogenic chemodosimeter that reacts with mercury ions at temperature ranges from about 0° C. to 100° C. allowing for detection of mercury ions.

One embodiment of the present invention is a method to detect mercury(II) ions in a sample. The method steps include:
preparing the sample by digestion methods to transform organic and sulfur-bound mercury species to sulfur-free inorganic mercury(II);
performing an oxymercuration reaction with the sample in the presence of about 0.01 μM to about 100 μM of a compound having a

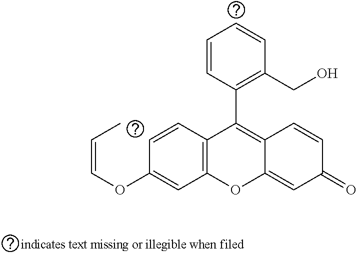

structure and about 0 mM to 100 mM of AgNO₃ in about 0% to about 5% DMSO in pH about 3 to about 7;

adjusting a temperature of the oxymercuration reaction between about 0° C. to about 100° C. and holding the temperature for about 0.1 hour to about 2 hours to create a resulting solution;

basifying the resulting solution to adjust the pH to between about 7 to about 11 to form a basified solution; and

measuring the fluorescence signals of the resulting solution.

The concentration of the compound can range from about 0.01 μM to about 100 μM, about 1 μM to about 75 μM, about 10 μM to 50 μM, about 20 μM to about 40 μM, or about 25 μM to about 35 μM.

The concentration of AgNO₃ can range from about 0 mM to about 100 mM, about 0.1 mM to about 90 mM, about 10 mM to about 80 mM, about 20 mM to about 60 mM, or about 30 mM to about 50 mM.

The temperature of the oxymercuration reaction can be adjusted between about 0° C. to about 100° C., about 0° C. to about 25° C., about 0° C. to about 85° C., about 10° C. to about 40° C., or about 20° C. to about 30° C.

The oxymercuration reaction can be performed from between about 0.1 hours and about 2 hours, about 0.25 hours and 1.5 hours, about 0.5 hours and 1.0 hours, and about 0.7 hours and 0.8 hours. The time at temperature must increase as the temperature decreases for an effective reaction with the compound to be greater than about 60% complete. For example, if the temperature is about 0° C., then the time at temperature is about 2 hours, and if the temperature is about 100° C., then the time at temperature is about 0.1 hours. See FIGS. 6A-C for relationship between reaction temperature and time at temperature.

Another embodiment of the present invention is a fluorogenic chemodosimeter having the following structure of:

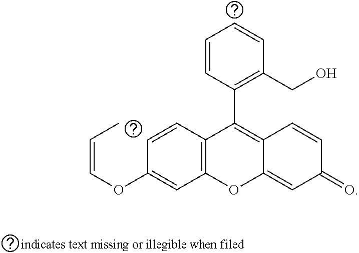

Another embodiment of the present invention includes the compound 12 with the following structure of:

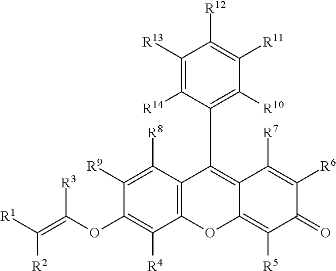

where:

R¹═H, alkyl, aryl, or other functional groups,

R²═H, alkyl, aryl, or other functional groups,

R³═H, alkyl, aryl, or other functional groups,

R⁴═H, alkyl, aryl, or other functional groups,

R⁵═H, alkyl, aryl, or other functional groups,

R⁶═H, alkyl, aryl, or other functional groups, but not Cl or F,

R⁷ and R⁸═H, alkyl, aryl, F, Cl, Br, aryl, or other functional groups,

R⁹═H, alkyl, aryl, or other functional groups, but not Cl or F,

R¹⁰ and R¹⁴═H, hydroxymethyl, alkyl, aryl, or other functional groups, excluding an electron-withdrawing group such as carboxylate, formyl, and ketone,

R¹¹ and R¹³═H, alkyl, aryl, F, Cl, Br, aryl, or other functional groups,

R¹²═H, alkyl, aryl, or other functional groups, excluding an electron-withdrawing group, wherein the electron-withdrawing group is selected from a group consisting of carboxylate, formyl, and ketone.

### BRIEF DESCRIPTION OF THE FIGURES

FIGS. 1A-D are results of the reaction to convert vinyl ether 3 to phenol 1 at 25° C.;

FIGS. 2A-B are the test results illustrating the compatibility of one embodiment of the present invention to detect Hg(II) in the presence of inorganic ions performed in pH 4 buffer;

FIGS. 3A-D are the test results of the applications of probe vinyl ether 3 for the detection of mercury species in environmental and dental samples;

FIG. 4A is a pH titration of compound 13 where fluorescence intensity at 515 nm was monitored at −0.3 pH intervals;

FIG. 4B is a UV-Vis absorption spectra of compounds 12 and 13 in 1% DMSO/pH 8 buffer;

FIG. 4C is an Emission spectra of compounds 12 and 13 in 0.1% DMSO/pH 8 buffer;

FIG. 5A is a pH-dependence comparison of reaction conditions for the conversion of 12 to 13 where fluorescence intensities were measured after addition of 1.3 M pH 7 buffer and 500 mM pH 10 buffer;

FIG. 5B is a Metal selectivity comparison of reaction conditions for the conversion of 12 to 13 where all the metals were tested at 5 μM in pH 4 buffer;

FIG. 5C is a Hg detection comparison of reaction conditions for the conversion of 12 to 13 in the presence of various metal ions where all of the reactions were performed in pH 4 buffer. Metal reagents: AgNO₃, AuCl₃, BaCl₂, CaCl₂, CdCl₂.2.5H₂O, CoCl₂, CrCl₃, CuCl₂.2H₂O, FeCl₃, HgCl₂, KCl, LiCl, MgCl₂, MnCl₂.4H₂O, NaCl, NiCl₂, Pb(NO₃)₂, Pd(NO₃)₂, PtCl₂, Rh(PPh₃)₃, RuCl₃, and ZnCl₂;

FIG. 5D is a correlation between fluorescence intensities at 515 nm and [Hg(II)] of reaction conditions for the conversion of 12 to 13 where the experiments were performed at pH 4 in triplicate;

FIG. 5E is a comparison of the effect of anion of reaction conditions for the conversion of 12 to 13 where all detection was performed in pH 4 buffer;

FIG. 6A is a comparison of reactive fluorescence (Time-course of the oxymercuration reaction at 25° C. in 50 mM phthalate pH 4.0 buffer) between compounds 12 and 14 where [probe]=1.0 μM, [Hg(II)]=0.3 μM;

FIG. 6B is a comparison of reactive fluorescence (Time-course of the oxymercuration reaction at 25° C. in 50 mM phthalate pH 4.0 buffer) between compounds 3 and 12 where [probe]=1.0 μM, [Hg(II)]=0.3 μM where fluorescence measurements were carried out every 1 min for 18 min.;

FIG. 6C is a comparison of reactive fluorescence (Time-course of the oxymercuration reaction in 50 mM phosphate pH 7 buffer) between compounds 3 and 12 where [probe]=1.0 μM, [Hg(II)]=0.3 μM;

FIG. 7 is a measurement of relative fluorescence of probe compound 12 applied for the detection of Hg(II) in river water where [compound 12]=1.0 μM; [Hg(II)]=0, 0.25, 0.5, 1, 2, and 4 ppb; [AgNO₃]=2.0 mM; 0.5% DMSO in 50 mM phthalate pH 4 water; 1 h; 25° C.;

FIG. 8A is a plot of UV-Vis absorption of compounds 1 and 3 in 0.1% DMSO/pH 7 buffer where absorption spectra were acquired on a Cary 50 UV/Vis spectrometer under the control of Windows-based PCs running the manufacturer's supplied software;

FIG. 8B is a plot of fluorescence emission spectra of compounds 1 and 3 in 0.1% DMSO/pH 7 buffer;

FIG. 8C is a plot of fluorescence emission spectra of compounds 13 and 12 in 0.1% DMSO/pH 8 buffer (excited at 470 nm to avoid the scattering);

FIG. 9A is a HPLC chromatogram of vinyl ether 3, where the HPLC analysis was performed on a Zorbax XDB C₁₈ column, 4.6×75 mm. Elution conditions: 1.0 mL/min, 60% MeOH/H₂O to 100% MeOH/H₂O linear gradient elution from 0 to 20 min;

FIG. 9B is a HPLC chromatogram of compound 2, where the HPLC analysis was performed on a Zorbax XDB C₁₈ column, 4.6×75 mm. Elution conditions: 1.0 mL/min, 60% MeOH/H₂O to 100% MeOH/H₂O linear gradient elution from 0 to 20 min;

FIG. 9C is a HPLC chromatogram of compound of a coinjected sample, where the HPLC analysis was performed on a Zorbax XDB C₁₈ column, 4.6×75 mm. Elution conditions: 1.0 mL/min, 60% MeOH/H₂O to 100% MeOH/H₂O linear gradient elution from 0 to 20 min;

FIG. 10A is a fluorescence response to Hg(II) applying vinyl ether 3 to various concentration of Hg, where vinyl ether 3=1.0 mM, [Hg]=0, 1, 2, 4, 8, 16, 32 ppb;

FIG. 10B is a fluorescence response to Hg(II) applying compound 12 to various concentration of Hg, where compound 12=1.0, [Hg]=0, 1, 2, 4, 8, 16, 32 ppb;

FIG. 11 is a plot that illustrates the relationship between fluorescence intensity and the concentration of compound 13 in 1% DMSO/50 mM phosphate pH 8 buffer were measured at various concentrations (0.78, 1.56, 3.13, 6.25, 12.5, 25, 50, 100, 200, and 400 nM) of 13, fluorescence Intensity=14670·[13]+66368. R²=0.9974;

FIGS. 12A-B are analyses of the data shown in FIG. 4a based on the following equation (pH=pKa−log [(Fₘₐₓ−F)/(F−Fₘᵢₙ)]), where the pKa values of compound 13 were 6.00 and 12.08;

FIG. 13 is a bar chart of Hg detection in the presence of Cl⁻ in a pH 4 buffer;

FIG. 14 is a ¹H NMR spectrum of vinyl ether 3 (300 MHz, acetone-d₆, 293K);

FIG. 15 is a ¹³C NMR spectrum of vinyl ether 3 (75 MHz, acetone-d₆, 293K);

FIG. 16 is a ¹H NMR spectrum of compound 8 (300 MHz, acetone-d₆, 293K);

FIGS. 17A-B are ¹H NMR spectrums of compound 10 (300 MHz, CDCl₃, 293K);

FIG. 18 is a ¹³C NMR spectrum of compound 10 (75 MHz, CDCl₃, 293K);

FIG. 19 is a ¹H NMR spectrum of compound 11 (300 MHz, acetone-d₆, 293K);

FIG. 20 is a ¹³C NMR spectrum of compound 11 (75 MHz, acetone-d₆, 293K);

FIG. 21 is a ¹H NMR spectrum of compound 12 (400 MHz, acetone-d₆, 293K);

FIG. 22 is a ¹³C NMR spectrum of compound 12 (100 MHz, acetone-d₆, 293K);

FIG. 23 is a ¹H NMR spectrum of compound 13 (300 MHz, acetone-d₆, 293K);

FIG. 24 is a ¹³C NMR spectrum of compound 13 (75 MHz, acetone-d₆, 293K);

FIG. 25 is a ¹H NMR spectrum of vinyl ether 3 before and after the treatment with HgCl₂ in the presence of NaCl (300 MHz, acetone-d₆, 293K); and

FIG. 26 is a bar chart with fluorescence intensities of one embodiment of the present invention to detect mercury in wastewater using a pretreatment of 10 N NaOH (500 μL) added to samples A and B from a coal-fired power plant (10 mL).

### DETAILED DESCRIPTION OF THE PREFERRED EMBODIMENT

As used herein in the specification and claims, including as used in the examples and unless otherwise expressly specified, all numbers may be read as if prefaced by the word “about”, even if the term does not expressly appear. Also, any numerical range recited herein is intended to include all sub-ranges subsumed therein.

The present invention, referred to herein as Scheme 2, will be compared with Scheme 1 to illustrate the benefits of the present invention.

Scheme 1 (shown below) is an allyl ether that is used as a protecting group and it can be removed via a base-catalyzed olefin migration followed by either an acid-catalyzed hydrolysis at elevated temperature or mercury-promoted hydrolysis (Scheme 1a—below). Scheme 1 including the following process steps: (a) Two-step sequence to cleave an allyl ether; (b) Platform for a fluorescence off-on switch; and c) Preparation of vinyl ether 3 and its reaction with HgCl₂ to form phenol 1.

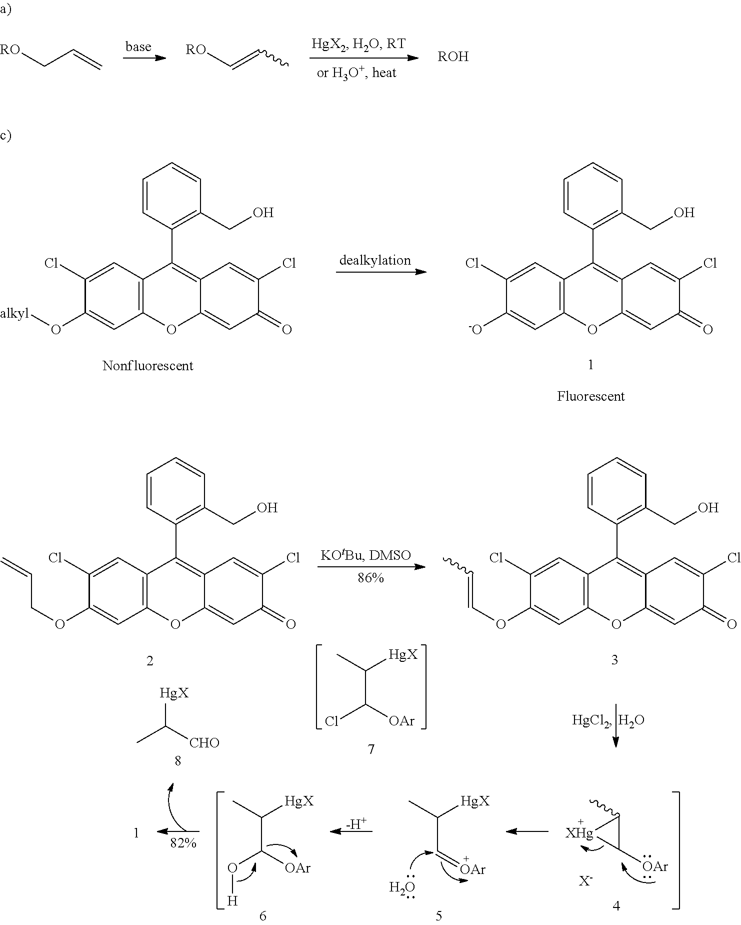

Because the oxymercuration of a vinyl ether can be employed at an ambient temperature, it is hypothesized that this transformation might be used as a means to selectively convert a nonfluorescent molecule to a fluorescent molecule with mercury ions.

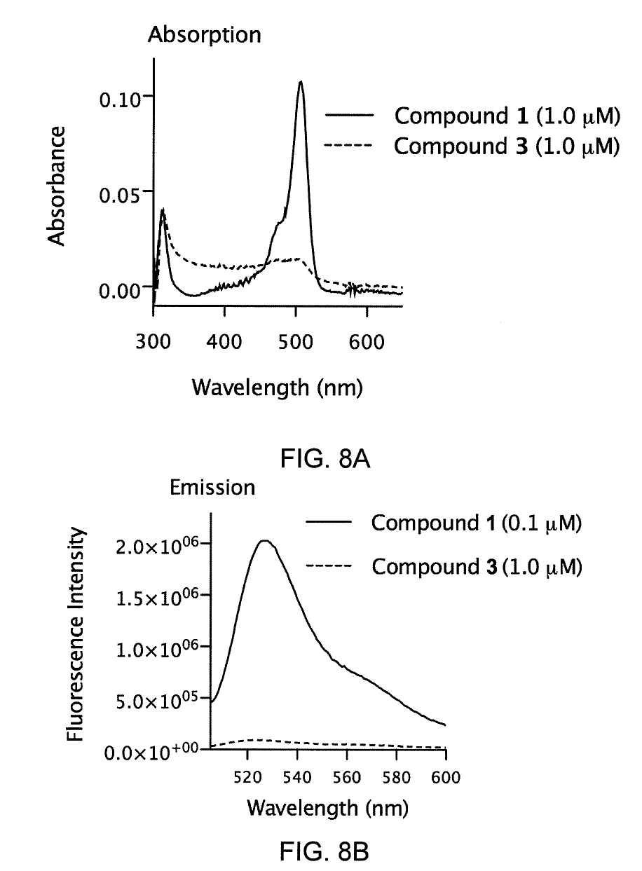

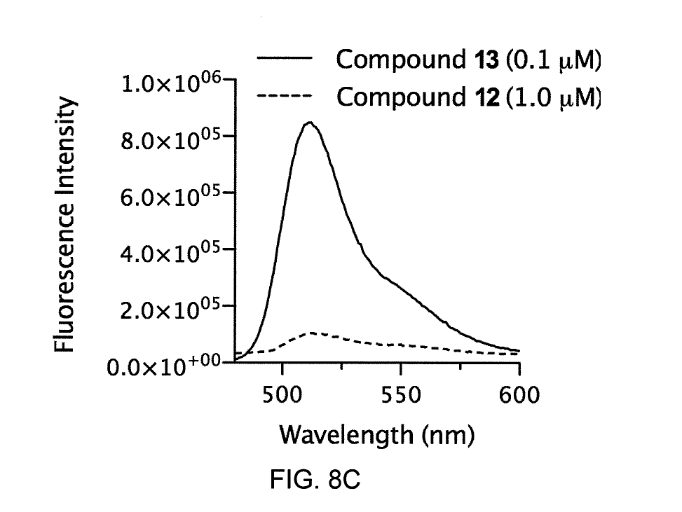

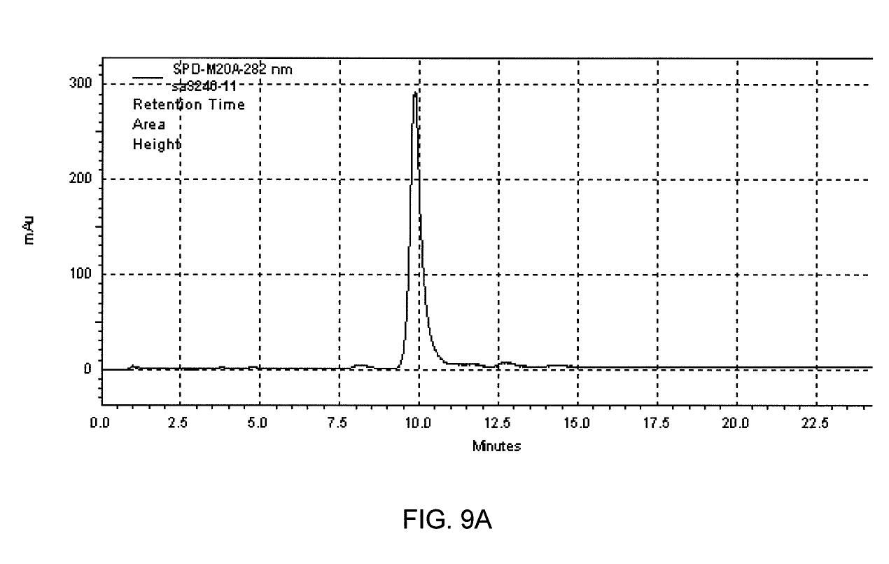

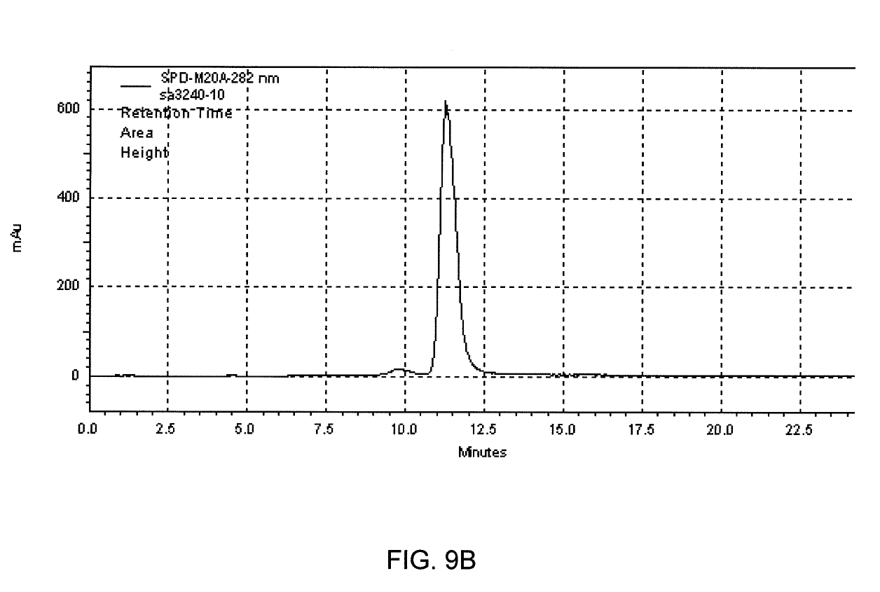

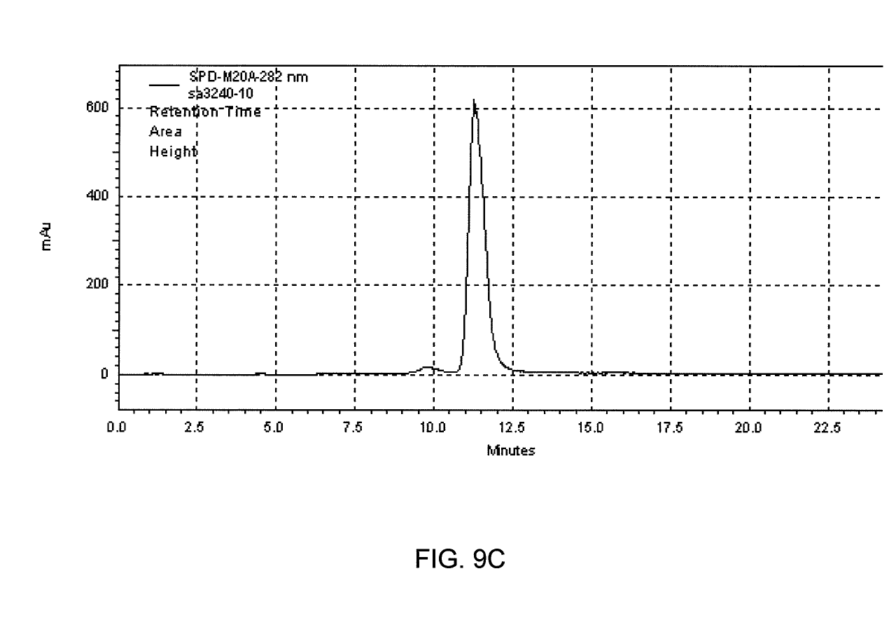

Scheme 1b (above) as a general platform for the development of fluorescence methods. In order to couple this platform with the chemistry shown in Scheme 1a, allyl ether 2 was treated with KOtBu in DMSO to form vinyl ether 3 in 86% yield as an inseparable mixture of cis and trans isomers (Scheme 1c above). The purity of vinyl ether 3 was ensured by HPLC analysis (FIGS. 9A-C). The absorption and emission spectra of vinyl ether 3 are shown in FIGS. 8A-C.

Treatment of vinyl ether 3 with HgCl₂ (1.0 equiv) at 25° C. afforded phenol 1 in 82% isolated yield, indicating that this transformation could be used to develop a fluorescence method for Hg(II) (possibly HgCl⁺, because HgCl₂ is mostly dissociated into HgCl⁺ and Cl⁻) at the more convenient temperature. Although vinyl ethers can be hydrolyzed under acidic conditions in a refluxing acetone-water mixture, such hydrolysis could not be detected when vinyl ether 3 was incubated in a pH 4 buffer at 80° C., ensuring its stability during storage.

Next, conditions for a Hg(II)-promoted hydrolysis of vinyl ether vinyl ether 3 to form phenol 1 were optimized in various buffers at 25° C.

TABLE 1
Buffer solutions were used as received from vendors.

| Buffer | Ingredients |
|---|---|
| pH 4 (50 mM) | potassium acid phthalate |
| pH 5 (50 mM) | potassium acid phthalate, sodium hydroxide |
| pH 6 (50 mM) | potassium acid phthalate, sodium hydroxide |
| pH 7 (50 mM) | potassium phosphate monobasic, sodium phosphate |
| | dibasic |
| pH 8 (50 mM) | sodium phosphate monobasic, potassium phosphate |
| pH 7 (1.23 M) | potassium phosphate monobasic, sodium hydroxide |
| pH 4 (250 mM) | potassium acid phthalate |
| pH 10 (500 mM) | boric acid, sodium hydroxide |

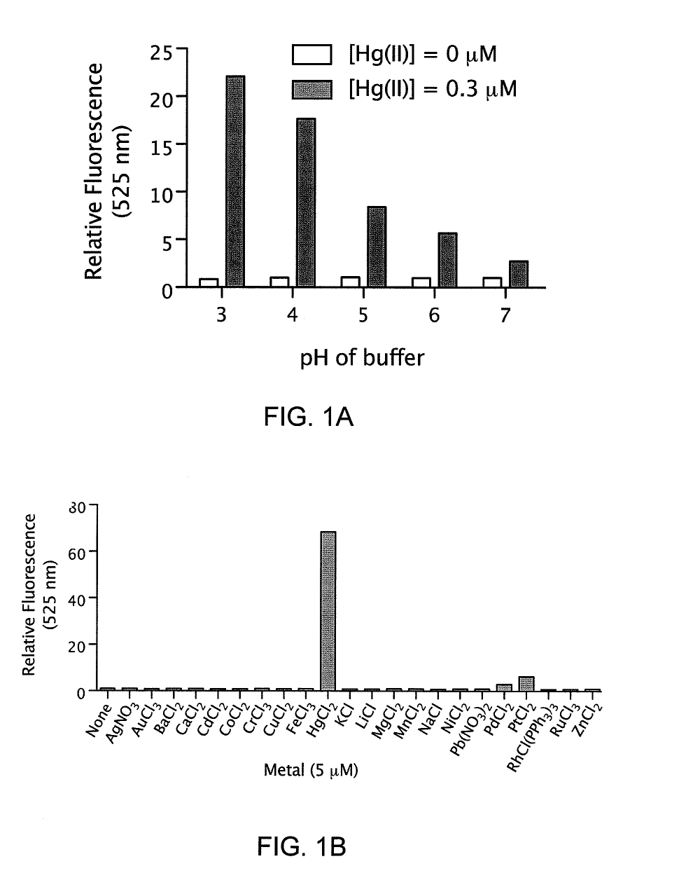

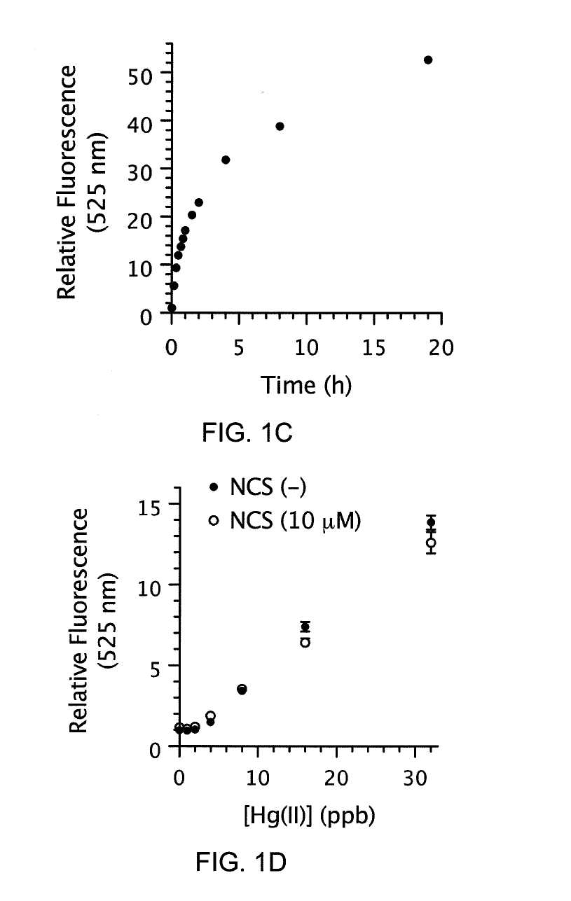

The ratio of Hg(II)-promoted and Hg(II)-free hydrolysis—the latter was negligible—was optimal at pH 3 (FIG. 1A). However, the pH 3 reaction media was more difficult to neutralize than the pH 4 media for fluorescence measurement in a high throughput manner. Therefore, pH 4 was chosen for the remaining experiments.

FIGS. 1A-D are the results of the reaction to convert vinyl ether 3 to phenol 1, where [vinyl ether 3]=1.0 μM for all of the experiments. Except for FIG. 1C, all of the reactions were performed for 1 h. Fluorescence intensities were measured after addition of 1.23 M phosphate pH 7 buffer. FIG. 1A is pH-dependence. FIG. 1B is metal selectivity in a 50 mM phthalate pH 4 buffer. Metal reagents: AgNO₃, AuCl₃, BaCl₂, CaCl₂, CdCl₂.2.5H₂O, CoCl₂, CrCl₃, CuCl₂.2H₂O, FeCl₃, HgCl₂, KCl, LiCl, MgCl₂, MnCl₂.4H₂O, NaCl, NiCl₂, Pb(NO₃)₂, Pd(NO₃)₂, PtCl₂, Rh(PPh₃)₃, RuCl₃, and ZnCl₂. FIG. 1C is time-course of the oxymercuration reaction. [Hg(II)]=0.30 μM, 50 mM phthalate pH 4 buffer. FIG. 1D is a correlation between fluorescence intensities and [Hg(II)] in the presence and absence of N-chlorosuccinimide (NCS). The experiments were performed in a 50 mM phthalate pH 4 buffer in triplicate. The graph shows the mean values and standard deviations.

In a pH 4 buffer, vinyl ether 3 was subjected to various metal ions at 5 μM, demonstrating that the conversion of vinyl ether 3 to phenol 1 was most efficiently promoted by Hg(II) (See FIG. 1B and Table 2). A slight signal increase was observed in the presence of Pt(II).

TABLE 2
Raw data for FIG. 1B (Metal selectivity)

| Metal | I₅₂₅ Metal |
|---|---|
| No metal | 125,186 |
| Ag | 123,852 |
| Au | 120,083 |
| Ba | 123,293 |
| Ca | 122,618 |
| Cd | 113,269 |
| Co | 117,022 |
| Cr | 126,897 |
| Cu | 120,330 |
| Fe | 123,457 |
| Hg | 8,555,110 |
| K | 119,310 |
| Li | 120,050 |
| Mg | 131,012 |
| Mn | 123,062 |
| Na | 120,017 |
| Ni | 123,392 |
| Pb | 121,170 |
| Pd | 384,773 |
| Pt | 781,743 |
| Rh | 94,441 |
| Ru | 119,260 |
| Zn | 123,375 |

I₅₂₅ = fluorescence intensity at 525 nm

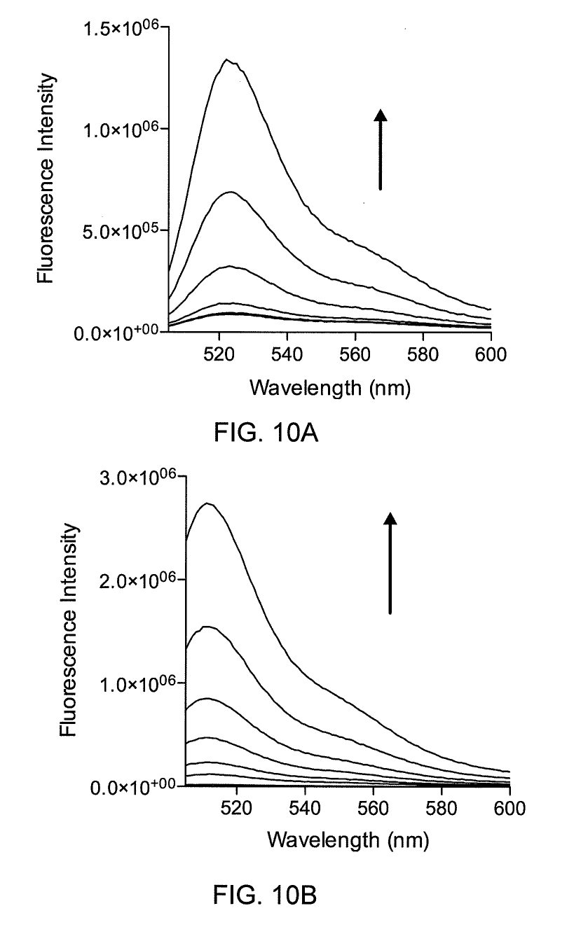

The conversion of vinyl ether 3 to phenol 1 in the presence of Hg(II) (0.30 μM) in a pH 4 buffer showed ˜40% completion after 1 h, and the reaction continued to proceed (FIG. 1C). The one-hour duration was considered an optimal balance between sensitivity and convenience. The fluorescence signals generated by the conversion of vinyl ether 3 to phenol 1 was [Hg(II)] dependent with a distinct linear signal beginning as low as 4 ppb (=20 nM) Hg(II) ( in FIG. 1D; FIGS. 10A-B and Table 3).

Oxidative pretreatment of environmental samples with Br—Cl is a standard procedure by the US EPA. It was found that NCS could also oxidatively disrupt the Hg—S bond. Because NCS can react with olefins, there was a concerned about the stability of vinyl ether 3 toward NCS. However, this probe was stable against NCS while remaining responsive to Hg(II) (O in FIG. 1D and Table 3). These data indicate that vinyl ether 3 could be used to detect Hg(II) in sulfur-containing samples in the presence of NCS.

TABLE 3
Raw data for FIG. 1D (Correlation between fluorescence
intensities and [Hg(II)] in the presence and absence of NCS)

| | Fluorescence Intensity at 525 nm | | | Fluorescence Intensity at 525 nm | | |
|---|---|---|---|---|---|---|
| | NCS (0 μM) | | | NCS (10 μM) | | |
| Hg(II) | Experiment | Experiment | Experiment | Experiment | Experiment | Experiment |
| (ppb) | 1 | 2 | 3 | 1 | 2 | 3 |
| 0 | 93,437 | 95,214 | 91,939 | 113,500 | 118,865 | 90,655 |
| 1 | 88,367 | 87,989 | 95,099 | 115,903 | 97,864 | 86,079 |
| 2 | 98,127 | 94,984 | 98,868 | 123,178 | 108,727 | 105,237 |
| 4 | 148,359 | 139,291 | 131,736 | 194,461 | 174,364 | 164,390 |
| 8 | 325,719 | 325,374 | 318,313 | 350,621 | 325,686 | 316,996 |
| 16 | 723,429 | 687,763 | 667,157 | 628,281 | 592,269 | 584,781 |
| 32 | 1,306,980 | 1,329,130 | 1,250,040 | 1,248,280 | 1,134,040 | 1,161,480 |

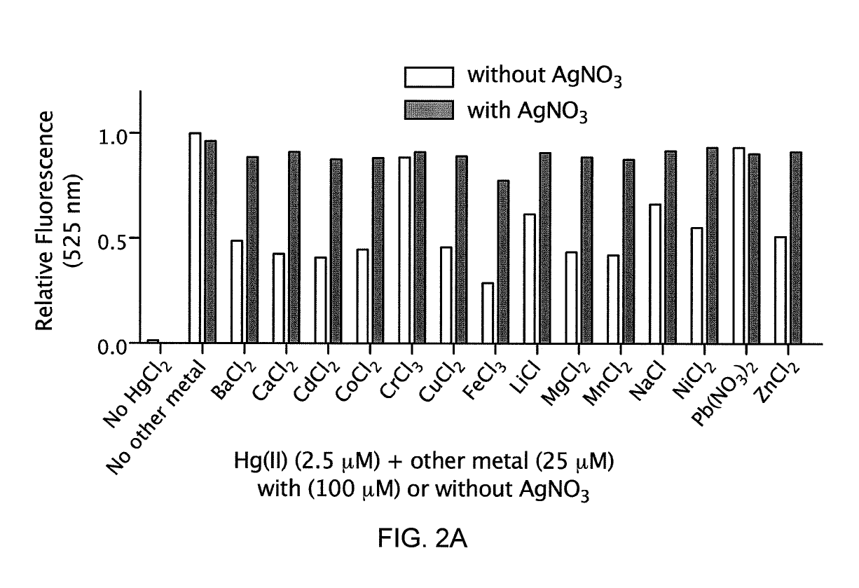

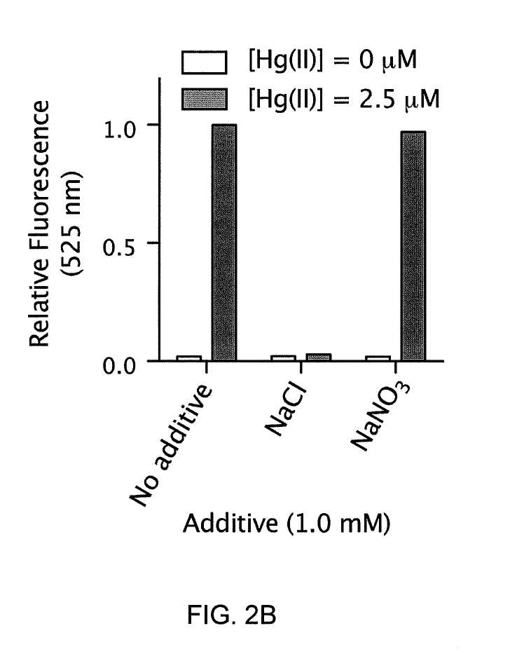

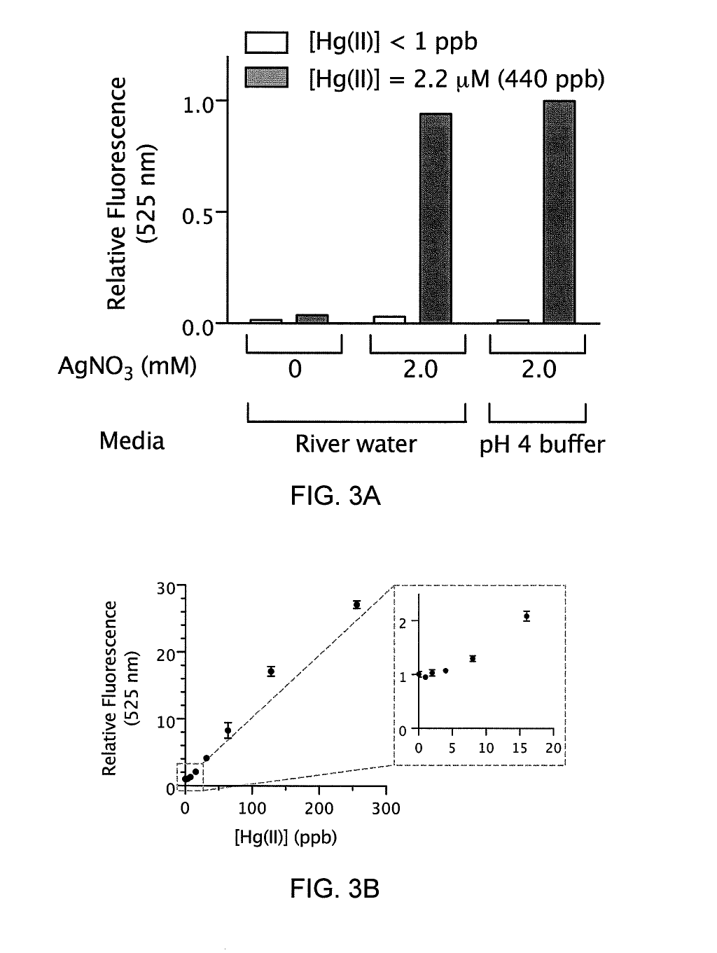

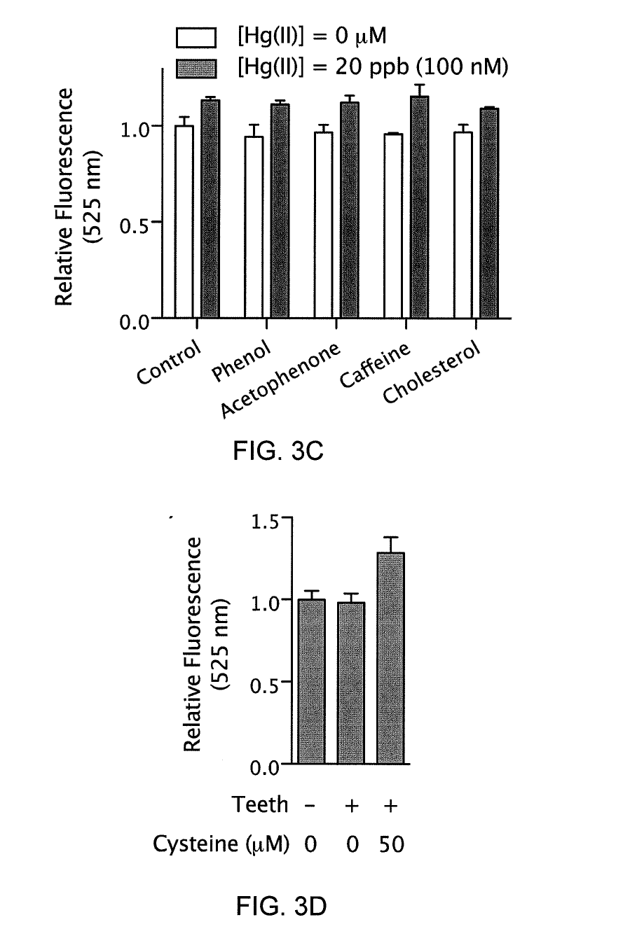

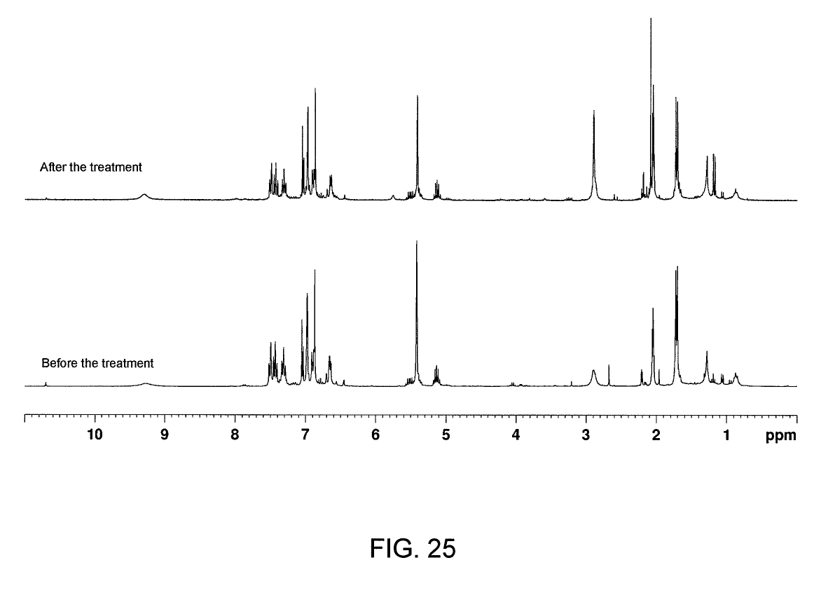

Now turning to FIG. 2A that illustrates interference levels by inorganic materials ([HgCl₂]=2.5 μM, [reagent shown]=25 μM), and FIG. 2B that illustrates the interference levels by NaCl with and without additive NaNO₃. Known amounts of Hg(II) were spiked into river water that contained <1 ppb total mercury to create actual “dirty” samples in order to evaluate the utility of the methods described herein. The resulting solutions, after adjusting the pH to 4, were treated with vinyl ether 3 in an attempt to convert vinyl ether 3 to phenol 1 but to no avail (FIG. 3A). Rather than testing each metal separately, mixtures of HgCl₂ (2.5 μM) with various inorganic reagents (25 μM) were examined. FIG. 2A (white bars) shows that most reagents interfered with the conversion of vinyl ether 3 to phenol 1. It appeared that this interference was not caused by metals but by chloride ions. A comparison of the effect of NaCl and NaNO₃ to verify the effect of chloride ions was conducted. Treatment of vinyl ether 3 (1.0 μM) with HgCl₂ (2.5 μM) and excess NaCl (1.0 mM) in a pH 4 buffer showed no fluorescence increase (FIG. 2b). Under similar reaction conditions, only the starting material vinyl ether 3 was observed by ¹H NMR spectroscopy (FIG. 25). In contrast, the presence of NaNO₃ did not impact the conversion of vinyl ether 3 to phenol 1. It is concluded that chloride ions interfered with the fluorescence method. This may also account for the failure of the above discussed river-water study.

Experiments were conducted on how chloride ions interfere with the Hg(II)-promoted conversion of vinyl ether 3 to phenol 1. The formation of compound 7 from the electrophilic species compound 5 (Scheme 1C) could be ruled out by the aforementioned NMR analysis. It is possible that the equilibrium shifted from more reactive HgX⁺ (X═Cl, phosphate, etc.) to less reactive HgXCl. This working hypothesis could account for the noninterference of the mixture of HgCl₂ and AgNO₃ (not shown). These results lead to the hypothesize that the addition of excess AgNO₃ to the mixtures of HgCl₂ and MClₙ (M=Li, Na, etc.) might facilitate the conversion of HgCl₂ (XHg—Cl bond: 24 kcal/mol) to HgCl⁺ by virtue of the formation of poorly water-soluble AgCl³⁸ (Ag—Cl bond: 71.7 kcal/mol). In effect, the combination of HgCl₂ (2.5 μM), AgNO₃ (100 μM), and MClₙ (25 μM) generated a nearly uniform fluorescence signal (FIG. 2A, gray bars). Thus, the addition of AgNO₃ was found to afford a more general fluorometric detection method for Hg(II) in the presence of various inorganic molecules.

Environmental samples were tested with the addition of AgNO₃ to detect mercury species. In wastewater, the permitted discharge limits for total mercury may be 5 ppb or 10 ppb. Thus, as a proof-of-concept experiment, one embodiment of the present invention involved the use of AgNO₃ for the detection of spiked Hg(II) (0-256 ppb) in river water (FIGS. 3A and 3B). FIG. 3A shows that unlike the previously failed case, the addition of AgNO₃ improves the signal recovery to nearly 100%. The standard curve in FIG. 3B (also Table 4) indicates that vinyl ether 3 could detect Hg(II) at −8 ppb in river water. All detection was performed after adjustment of pH of samples to pH 4.

FIG. 3A is a comparison of river water and commercial pH 4 buffer. [HgCl₂]=2.2 μM (440 ppb). In the absence of AgNO₃ (0 mM), Hg(II) cannot be detected by the method (FIG. 3A-left bar charts). In the presence of AgNO₃ (2 mM), Hg(II) can be detected (FIG. 3A-middle bar charts). FIG. 3B is mercury detection in river water. [HgCl₂]=0-256 ppb, [AgNO₃]=2.0 mM. FIG. 3C is mercury detection in river water in the presence of organic compound. [AgNO₃]=2.0 mM, [compound]=100 ppb. FIG. 3D are Dental samples. [NCS]=500 μM.

TABLE 4
Raw data for FIG. 3B (mercury detection in river water)
Fluorescence Intensity at 525 nm

| Spiked Hg(II) (ppb) | Experiment 1 | Experiment 2 | Experiment 3 |
|---|---|---|---|
| 0 | 314,231 | 298,957 | 357,304 |
| 1 | 309,145 | 293,987 | 316,651 |
| 2 | 369,187 | 310,034 | 317,556 |
| 4 | 362,587 | 327,612 | 345,322 |
| 8 | 451,415 | 404,145 | 399,274 |
| 16 | 731,000 | 640,675 | 647,028 |
| 32 | 1,428,440 | 1,314,600 | 1,280,670 |
| 64 | 2,939,690 | 2,846,650 | 2,256,380 |
| 128 | 5,364,490 | 5,814,890 | 5,463,050 |
| 256 | 8,949,830 | 8,803,740 | 8,593,100 |

The above discussed method in the presence of various typical organic contaminants in wastewater was used to assess the robustness of vinyl ether 3 with other functional groups (FIG. 3C and Table 5) to determine whether the method based on the oxymercuration of vinyl ether vinyl ether 3 could be compatible with organic contaminants in environmental samples. It was found that this probe detected Hg(II) even if the reaction solution was contaminated with organic compounds, including an alkene, because the olefin of vinyl ether 3 is more electron rich and thus more reactive toward Hg(II) than most alkenes.

TABLE 5
Raw data for FIG. 3C (mercury detection in river water in
the presence of organic compound) Fluorescence Intensity at 525 nm

| | Hg(−) | | | Hg(+) | | |
|---|---|---|---|---|---|---|
| Contaminant | Experiment 1 | Experiment 2 | Experiment 3 | Experiment 1 | Experiment 2 | Experiment 3 |
| — | 247,721 | 228,629 | 229,633 | 267,340 | 262,732 | 270,796 |
| Phenol | 235,097 | 225,650 | 205,965 | 261,793 | 256,543 | 267,291 |
| Acetophenone | 237,402 | 227,724 | 217,980 | 254,733 | 269,068 | 270,270 |
| Caffeine | 226,572 | 224,432 | 225,535 | 256,165 | 274,582 | 284,786 |
| Cholesterol | 238,323 | 226,572 | 218,918 | 257,103 | 258,913 | 255,259 |

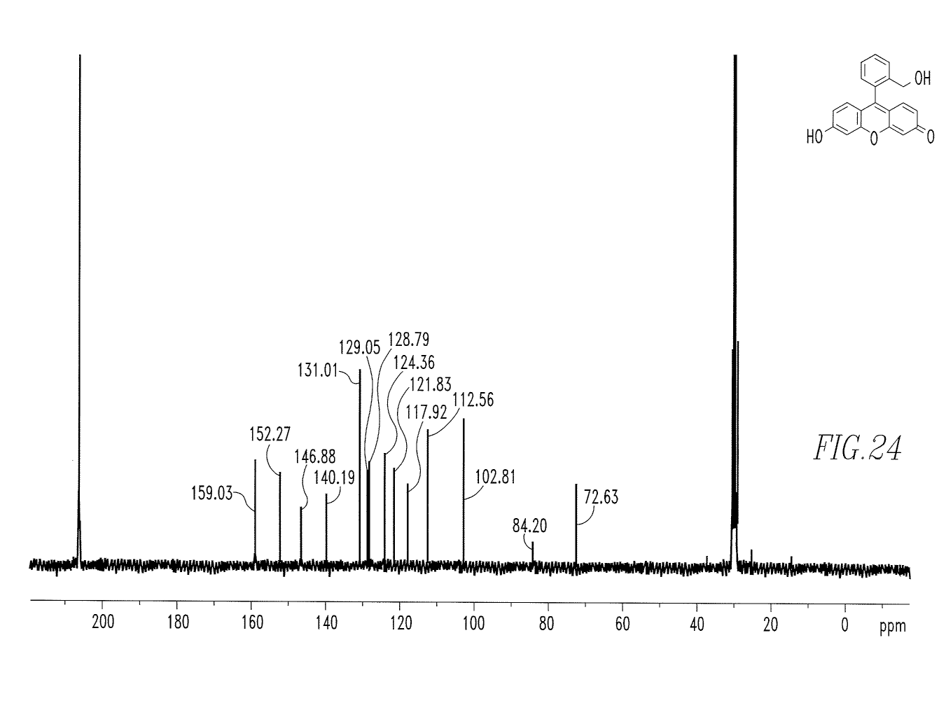

In addition to environmental samples, dental samples were examined to broaden the applications of the fluorescence method. It is hypothesized that cysteine from food might facilitate the dissolution of Hg from amalgam-filled teeth. Thus, previously used teeth were stirred in a cysteine solution. After the teeth were removed, the resulting solution was treated with NCS to oxidize the thiol and Hg-bound sulfur atoms before the addition of vinyl ether 3. FIG. 3D and Table 6 show that one embodiment of the present invent can detect, although unable to quantify, leached mercury from dental samples. The difference between bars 2 and 3 (3.63×10⁴ in the raw data; see FIG. 24) was greater than three times the standard deviation of bar 2 (6.85×10³), enabling us to detect Hg(II) with a confidence level of over 90%. Additional studies of dental samples are needed to validate the utility of the method in dentistry. Nonetheless, this result may warrant further studies on how sulfur-rich food may dissolve mercury amalgams.

TABLE 6
Raw data for FIG. 3D (dental samples)
Fluorescence Intensity at 525 nm

| Conditions | Experiment 1 | Experiment 2 | Experiment 3 |
|---|---|---|---|
| H₂O only | 114,306 | 126,683 | 120,758 |
| Teeth with H₂O | 113,335 | 116,018 | 126,305 |
| Teeth with cysteine solution | 167,123 | 144,031 | 153,544 |

It should be noted that vinyl ether 3 cannot be contaminated with heavy metals. It reacts with Hg(II) in a 1:1 stoichiometry, and anion interference and a solution to this interference were discovered.

Scheme 2 was developed to circumvent interference by chloride ions, although organic and inorganic chlorides are not the same. Further consideration on removing the chlorides from vinyl ether 3 suggested that the vinyl ether of compound 12 (Scheme 2 below) might be more reactive toward Hg(II) due to the lack of electron-withdrawing chloride groups. Preparation of compound 12 and its reaction with HgCl₂ forms compound 13:

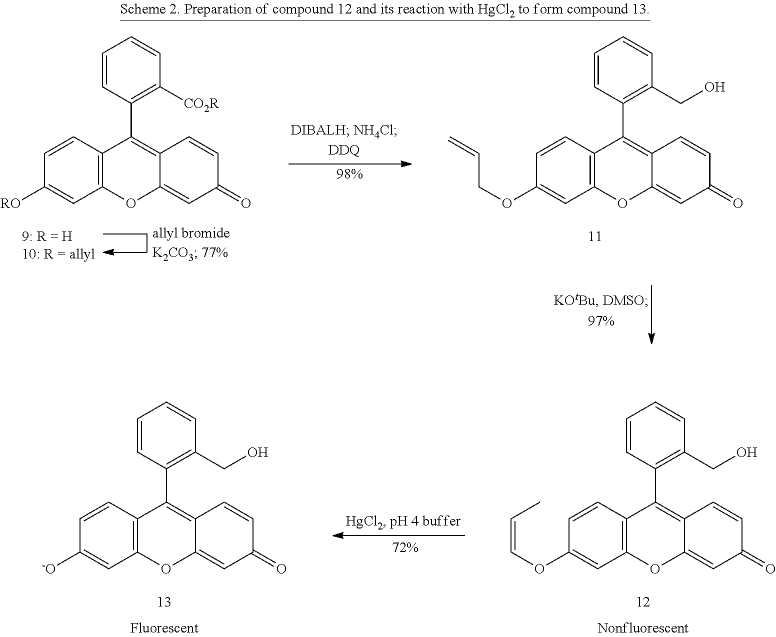

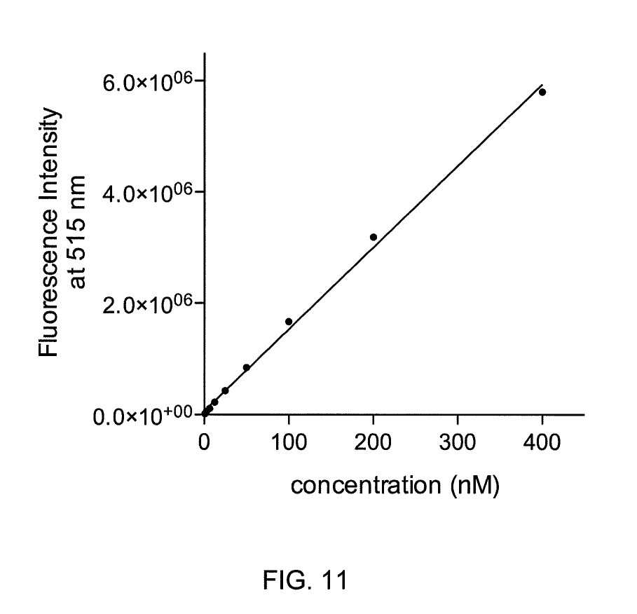

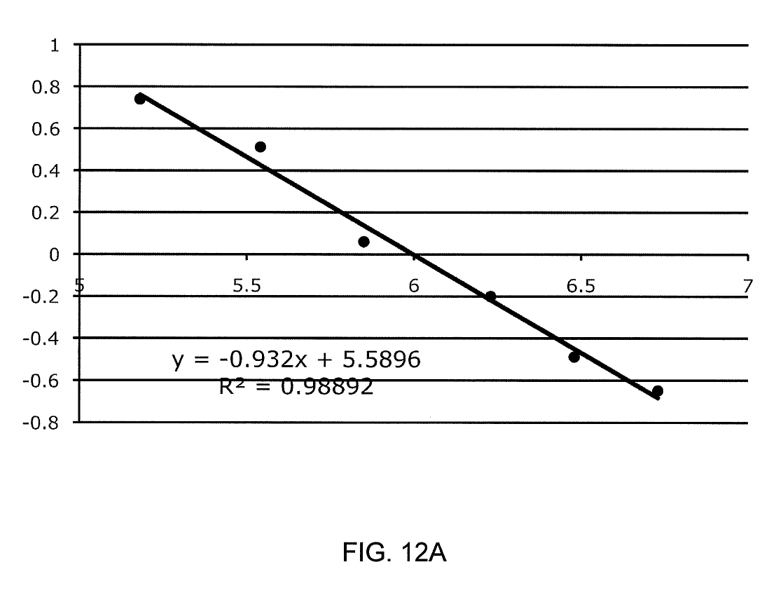

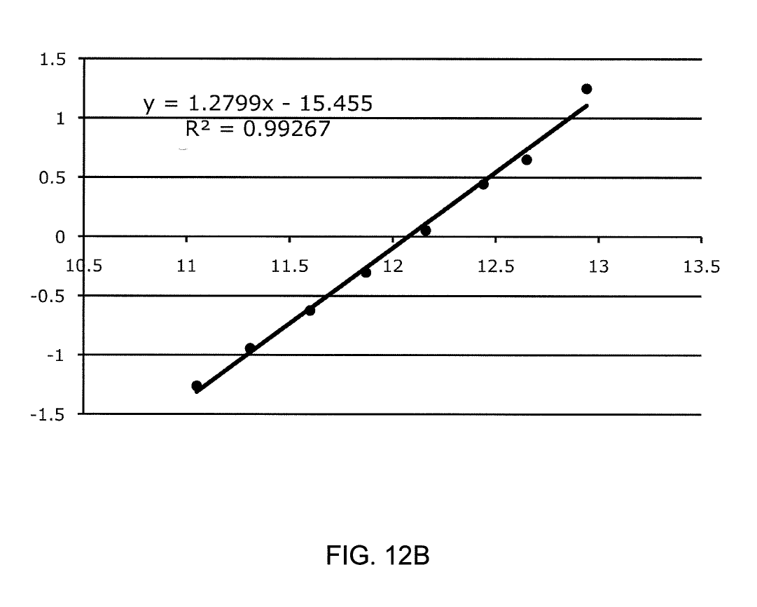

The olefin migration of the allyl ether compound 11 was highly stereoselective, only giving the cis product compound 12. Compound 12 also reacted with HgCl₂ smoothly at 25° C. and gave the new fluorescent compound 13 in 72% isolated yield. The linear correlation was confirmed between the concentration of compound 13 and the fluorescence signals (FIG. 11).

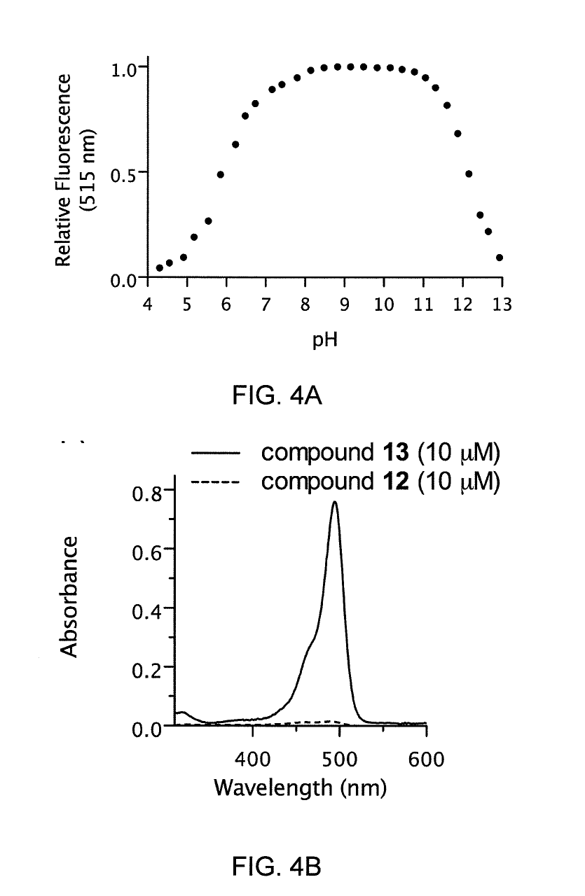

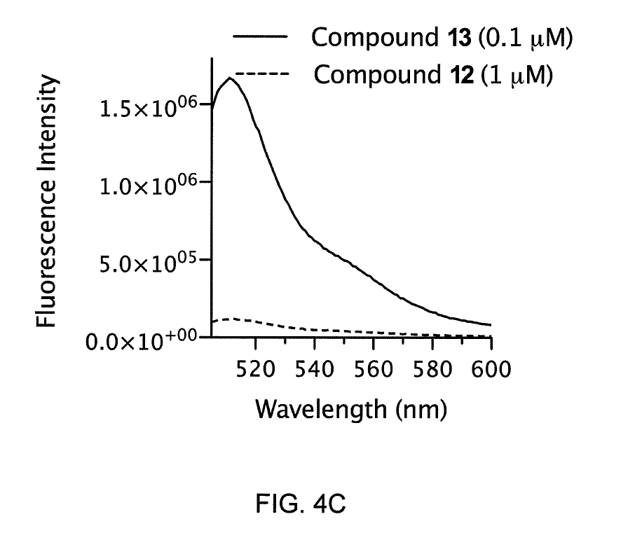

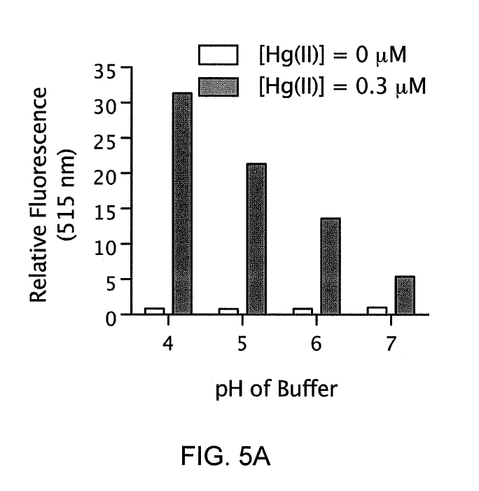

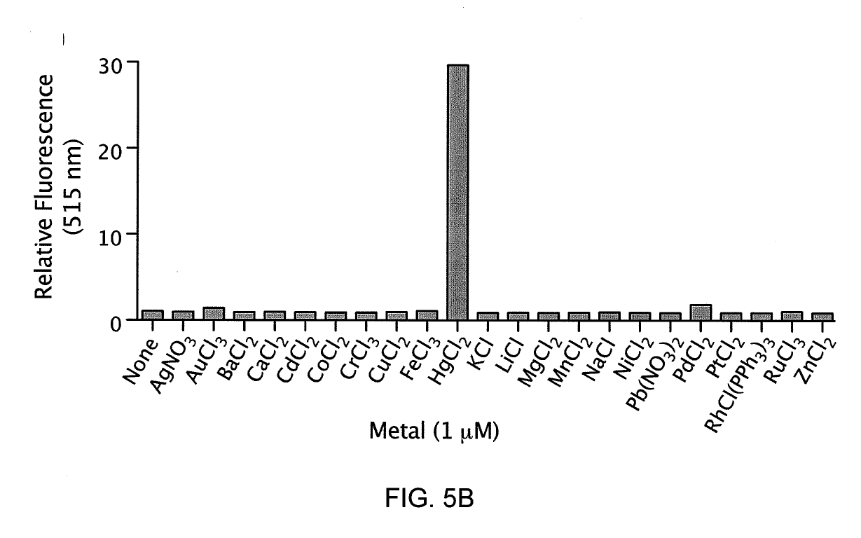

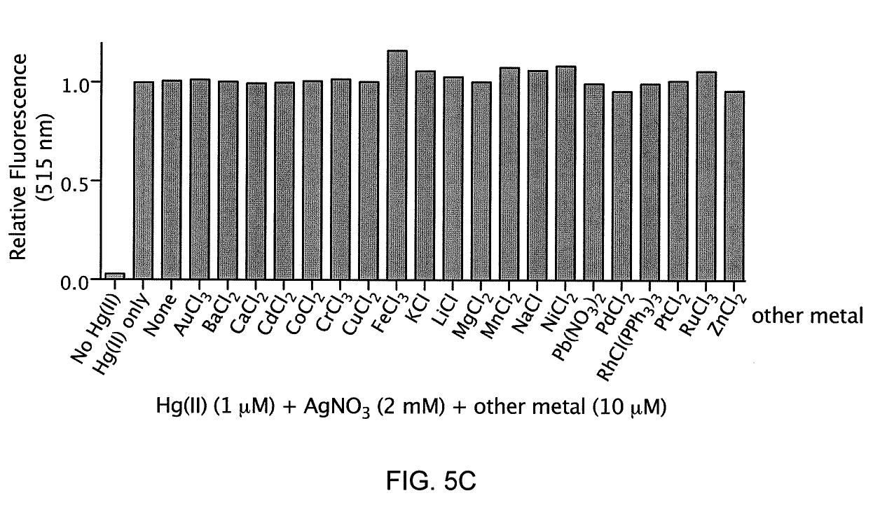

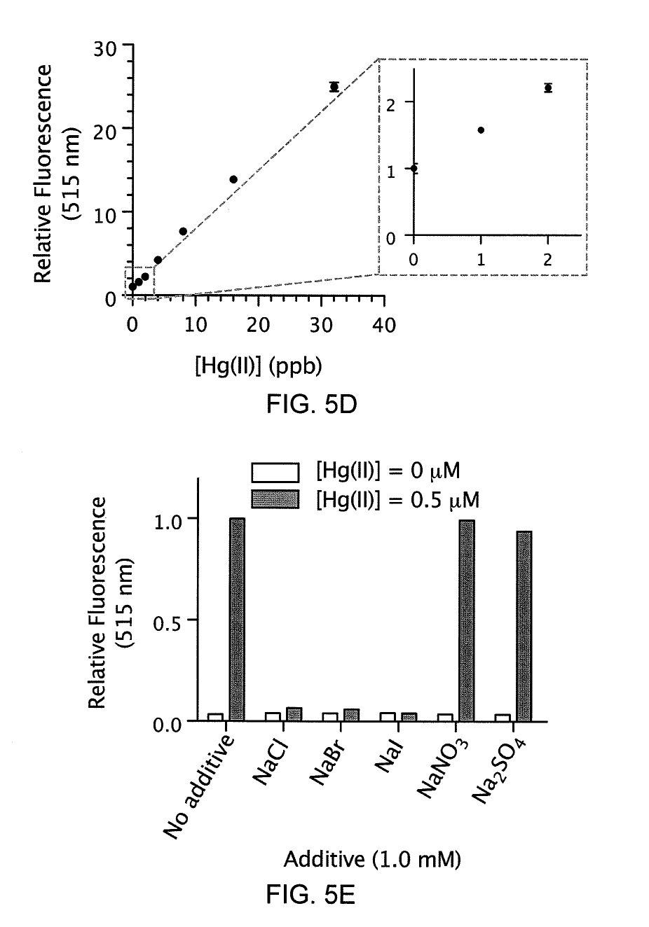

The pKₐ of the phenolic hydroxy group of compound 13 was 6.0 (FIG. 4A and FIGS. 5A-B), noticeably lower than that of fluorescein (6.4). This was unexpected because the conversion of the stronger electron-withdrawing carboxy group of fluorescein to the weaker hydroxymethyl group should decrease the acidity of the phenol hydroxy group. It is postulated that the carboxylate anion of fluorescein might destabilize the phenoxy anion intramolecularly. The fine tuning of the acidity of phenol in fluorescein derivatives can be used to develop new assay methods.

The UV-Vis absorption spectra of compounds 12 and 13 were obtained as shown in FIG. 4B. Compound 12 showed no absorption peak at ˜490 nm, but compound 13 did. The fluorescence emission spectra of compounds 12 and 13 indicated that the signal of compound 12 was 140 times lower than that of compound 13 in a pH 8 buffer at 515 nm (λₘₐₓ) at the same concentration (FIG. 4C; see also FIGS. 8A-C). This is consistent with the platform depicted in Scheme 1B.

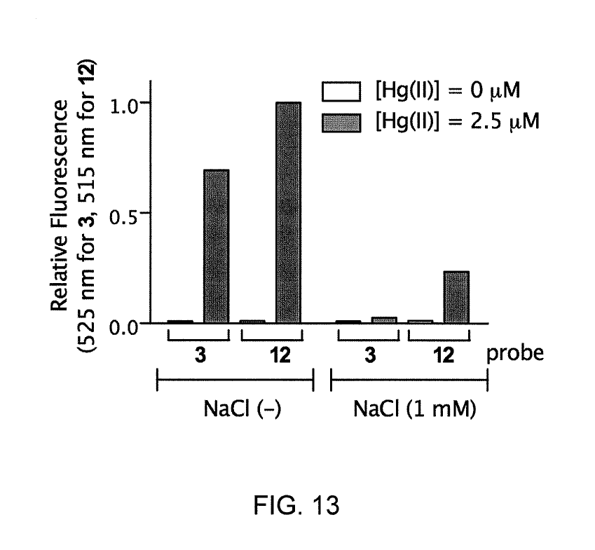

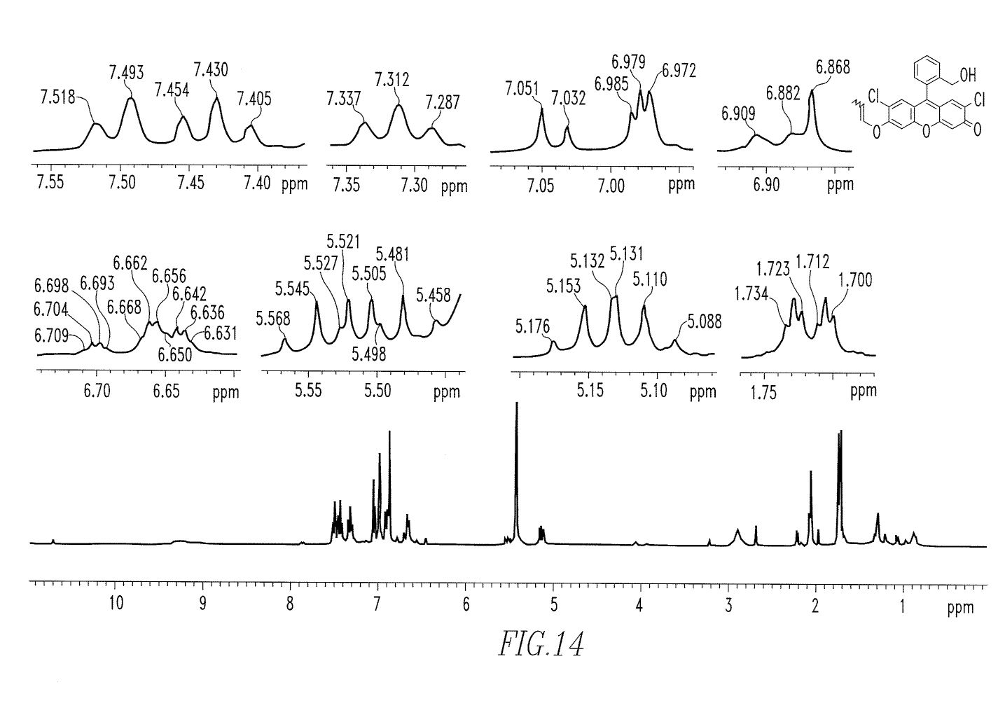

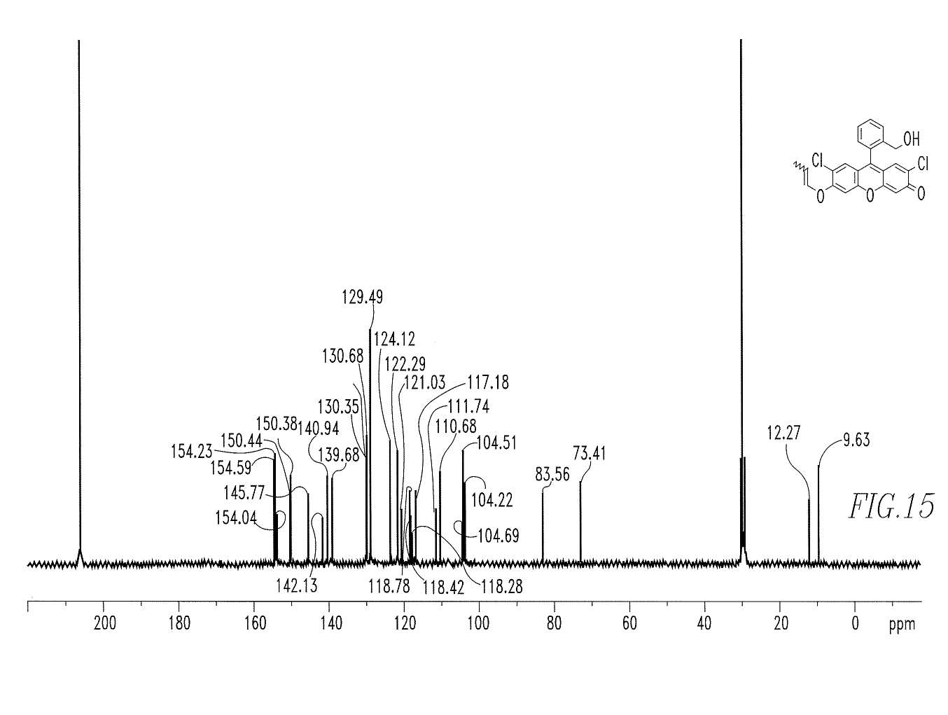

Solutions of the vinyl ether compound 12 were treated with Hg(II) (0.3 μM==60 ppb) in pH 4, 5, 6, and 7 buffer (FIG. 5A). Similarly to vinyl ether 3, compound 12 was more reactive at a lower pH. The reactivity of compound 12 toward Hg(II) was very high (FIG. 5B). The only other metal that reacted with compound 12 was Pd(II) (FIG. 1B). In the presence of AgNO₃, none of the coexisting metal reagents interfered with the Hg(II)-promoted conversion of compound 12 to compound 13 (FIG. 5C). It was found that the fluorescence method using compound 12 allowed for the detection of 1 ppb Hg(II) (FIG. 5D and Table 7). FIG. 5E indicates that chloride, bromide, and iodide ions interfered with the Hg(II)-promoted conversion of compound 12 to compound 13, but nitrate and sulfate ions did not. The interference by chloride ions was less severe for compound 12 than for vinyl ether 3 (FIG. 13). The halide-ion-mediated interference could be overcome by the addition of AgNO₃ (FIG. 5C).

TABLE 7
Raw data for FIG. 5D (Hg(II) vs. fluorescence with
compound 12) Fluorescence Intensity at 515 nm

| Hg(II) | | | |
|---|---|---|---|
| (ppb) | Experiment 1 | Experiment 2 | Experiment 3 |
| 0 | 114,469 | 106,823 | 98,594 |
| 1 | 166,000 | 168,000 | 170,000 |
| 2 | 243,103 | 229,753 | 234,419 |
| 4 | 450,877 | 448,544 | 442,885 |
| 8 | 819,761 | 810,176 | 817,535 |
| 16 | 1,480,390 | 1,468,780 | 1,491,300 |
| 32 | 2,611,540 | 2,673,740 | 2,723,060 |

Compound 12:

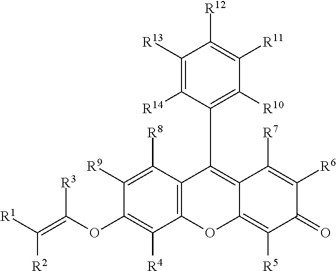

where:

R¹═H, alkyl, aryl, or other functional groups,

R²═H, alkyl, aryl, or other functional groups,

R³═H, alkyl, aryl, or other functional groups,

R⁴═H, alkyl, aryl, or other functional groups,

R⁵═H, alkyl, aryl, or other functional groups,

R⁶═H, alkyl, aryl, or other functional groups, but not Cl or F,

R⁷ and R⁸═H, alkyl, aryl, F, Cl, Br, aryl, or other functional groups,

R⁹═H, alkyl, aryl, or other functional groups, but not Cl or F,

R¹⁰ and R¹⁴═H, hydroxymethyl, alkyl, aryl, or other functional groups, excluding an electron-withdrawing group such as carboxylate, formyl, and ketone,

R¹¹ and R¹³═H, alkyl, aryl, F, Cl, Br, aryl, or other functional groups,

R¹²═H, alkyl, aryl, or other functional groups, excluding an electron-withdrawing group, wherein the electron-withdrawing group is selected from a group consisting of carboxylate, formyl, and ketone.

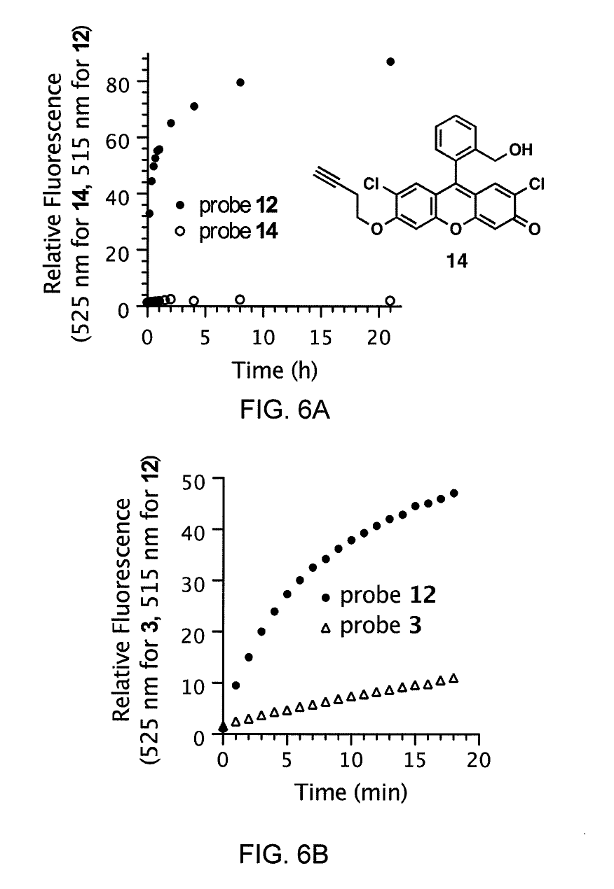

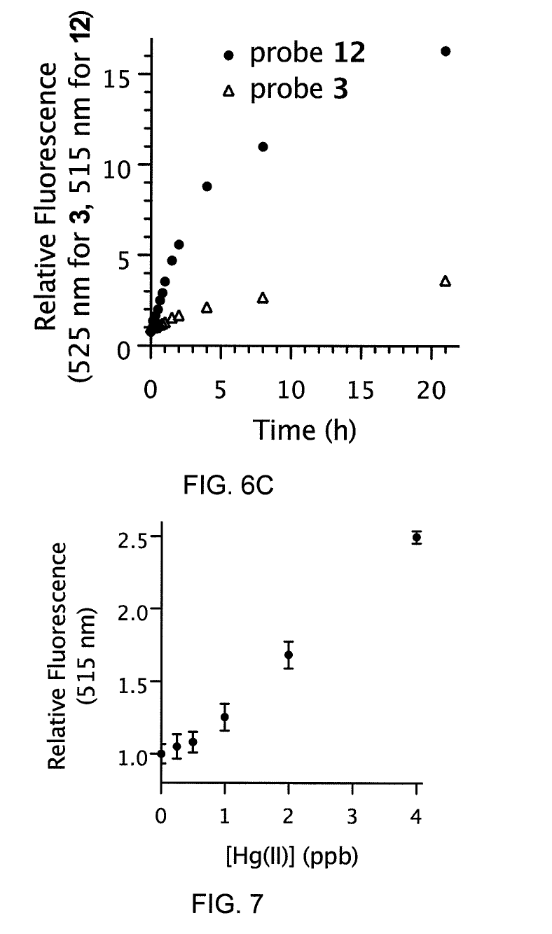

Next, the new chemodosimeter compound 12 is compared with compounds 3 and 14. As FIG. 6A shows, compound 12 was far more reactive than compound 14 toward Hg(II) in a pH 4 buffer at 25° C. For example, after 10 min the fluorescence increase with compound 12 was 242 times greater than that with compound 14. The reaction with compound 12 was >60% complete after 1 h. It is even more noteworthy that the initial rate was about 12 times greater for compound 12 relative to vinyl ether 3 (FIG. 6B). Additionally, compound 12 was more reactive than vinyl ether 3 at pH 7 (FIG. 6C), implying potential biological applications.

Chemodosimeter compound 12 was applied for detection of Hg(II) spiked in river water. Chemodosimeter compound 12 was able to detect 1 ppb Hg in river water (FIG. 7 and Table 8), which is below the US EPA's limitation for drinking water (2 ppb). The heightened sensitivity of compound 12 in determining [Hg(II)] in such a complex media as natural water indicates its robustness and potential for facile on-site monitoring of water safety. When the method of the present invention was applied to the analysis of two wastewater samples from a coal-fired power plant, compound 12 is a qualitative indicator of Hg(II) discussed in detail below. Compound 12 was able to qualitatively discriminate between concentration values as low as 2 ppb and <500 ppt.

TABLE 8
Raw data for FIG. 7 (Application of compound 12 for the
detection of Hg(II) in river water) Fluorescence Intensity at 515 nm

| Spiked Hg(II) (ppb | Experiment 1 | Experiment 2 | Experiment 3 |
|---|---|---|---|
| 0.0 | 124,473 | 137,218 | 120,960 |
| 0.25 | 128,482 | 126,975 | 146,351 |
| 0.50 | 146,892 | 129,047 | 137,446 |
| 1.0 | 172,501 | 151,219 | 154,673 |
| 2.0 | 218,897 | 223,030 | 201,118 |
| 4.0 | 320,437 | 311,611 | 321,717 |

The present invention is a sensitive and selective fluorometric method to detect mercury species at an ambient temperature in the presence of various organic, inorganic, and anionic contaminants. The method with vinyl ether 3 was effective in the detection of Hg(II) in river water and dental samples. Further structural fine-tuning led to the development of the vinyl ether compound 12.

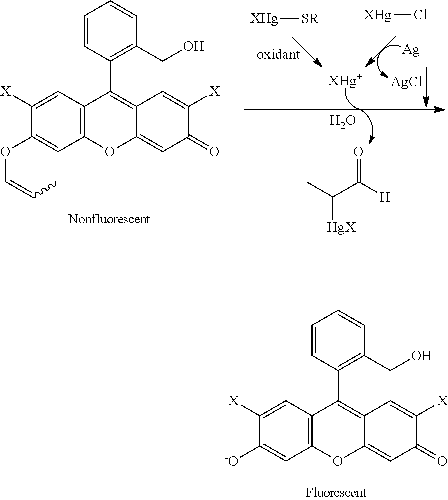

Compound 12 could react with Hg(II) after the removal of chloride ions with AgNO₃. This compound is 242 times more reactive the alkyne 14 toward Hg(II) and could be used to detect Hg(II).

Experimental Section

All of the reactions in Scheme 1c and 2 were carried out with commercial-grade reagents without further purification. DMF was used after distillation from silica gel. CH₂Cl₂ was used after distillation from CaH₂. Yields refer to chromatographically and spectroscopically (¹H NMR) homogeneous materials. All reactions were monitored by thin-layer chromatography (TLC) carried out on 0.25 mm E. Merck silica gel plates (60E-254) using UV light (254 nm) for visualization or phosphomolybdic acid in ethanol as developing agents and heat for visualization. Silica gel (230-400 mesh) was used for flash chromatography. NMR spectra were recorded on AM300 or AM400 (Bruker) instruments and calibrated using a solvent peak as an internal reference. The following abbreviations are used to indicate the multiplicities: s=singlet, d=doublet, t=triplet, q=quartet, m=multiplet, br=broad, app=apparent. High-resolution mass spectra were obtained using EBE geometry.

Vinyl ether 3 was prepared according to the following process. KOtBu (11 mg, 0.10 mmol) was added to a solution of compound 2 (21 mg, 50 μmol) in DMSO (1.0 mL) at 25° C. under a nitrogen atmosphere, and the resulting solution was heated in a 90° C. oil bath for 12 h. The reaction mixture was then poured onto ice-cold H₂O (5.0 mL), and the resulting mixture was extracted with EtOAc (2×5 mL). The combined organic layers were washed with brine (10 mL), dried over Na₂SO₄, filtered, and evaporated under reduced pressure. The residue was purified by flash column chromatography (5% to 20% EtOAc in hexanes) on silica gel (10 mL) to afford vinyl ether 3 (18 mg, 86%, mixture of cis/trans (2:1)) as an orange solid. Data for 3: m.p.=146-155° C.; R_{f}=0.31 (30% EtOAc in hexanes); IR (KBr pellet): νₘₐₓ=3368 (broad, O—H), 2921, 2859, 1606 (C═O), 1482, 1435, 1411, 1269, 1175, 1034, 874, 725 cm⁻¹; ¹H NMR (300 MHz, acetone-d₆, 293K, FIG. 14): δ=7.52-7.39 (m, 2H, Ar), 7.34-7.27 (m, 1H, Ar), 7.05 (s, 0.67H, Ar (cis isomer)), 7.03 (s, 0.33H, Ar (trans isomer)), 6.99-6.97 (m, 2H, Ar), 6.91-6.88 (m, 1H, Ar), 6.87 (s, 1H, Ar), 6.68 (dq, J=12.0, 1.8 Hz, 0.33H, CH₃CH═CHOAr (trans isomer)), 6.65 (dq, J=6.6 (A similar coupling constant was observed in a related compound⁴⁷), 1.8 Hz, 0.67H, CH₃CH═CHOAr (cis isomer)), 5.51 (dq, J=12.0, 6.6 Hz, 0.33H, CH₃CH═CHOAr (trans isomer)), 5.42 (s, 2H, ArCH₂OH), 5.13 (dq, J=6.6,⁴⁷ 6.6 Hz, 0.67H, CH₃CH═CHOAr (cis isomer)), 1.76-1.66 (m, 3H, CH₃CH═CHOAr) ppm; ¹³C NMR (75 MHz, acetone-d₆, 293K, FIG. 15): δ=154.6, 154.2, 154.0, 150.44, 150.38, 145.8, 142.1, 140.9, 139.7, 130.7, 130.4, 129.5, 124.1, 122.3, 121.0, 118.8, 118.4, 118.3, 117.2, 111.7, 110.7, 104.7, 104.5, 104.2, 83.6, 73.4, 12.3, 9.6 ppm; HRMS (EI+) m/z calcd. for C₂₃H₁₆O₄Cl₂ [M]⁺ 426.0426, found 426.0425.

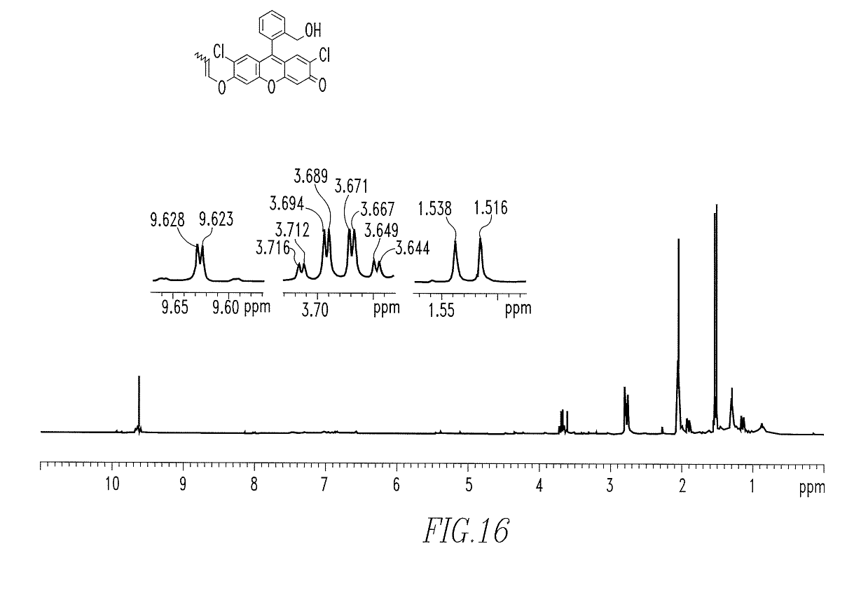

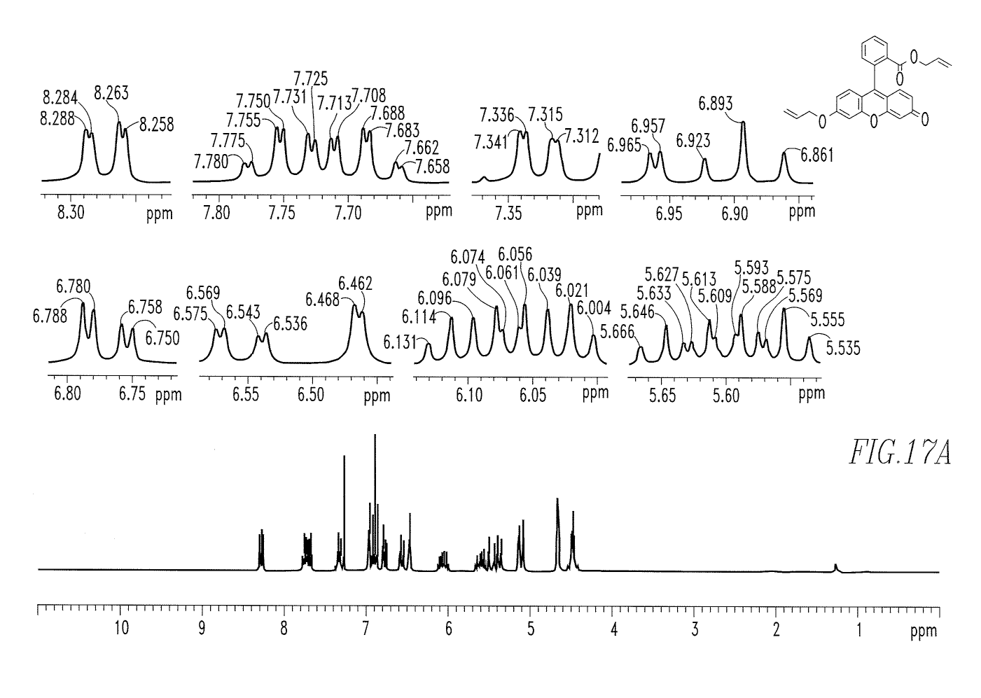

Vinyl ether 3 was converted to phenol 1 according to the following process. HgCl₂ (14 mg, 50 μmol) was added to a solution of vinyl ether 3 (21 mg, 50 mol) in a mixture of 1:9 DMSO/50 mM phthalate pH 4 buffer (20 mL). The resulting mixture was stirred at 25° C. for 1 h and extracted with EtOAc (2×20 mL). The combined organic layers were washed with brine (2×30 mL), dried over Na₂SO₄, filtered, and evaporated under reduced pressure. The residue was purified by flash column chromatography (10% to 70% EtOAc in hexanes) on silica gel (10 mL) to afford phenol 1 (16 mg, 82%) as an orange solid. The spectroscopic data of phenol 1 were consistent with the literature. Compound 8 was isolated from other fractions. R_{f}=0.66 (50% EtOAc in hexanes); ¹H NMR (300 MHz, acetone-d₆, 293K, FIG. 16): δ=9.63 (d, J=1.2 Hz, 1H, CHO), 3.66 (qd, J=6.9, 1.2 Hz, 1H, OHC—CHHgX), 1.53 (d, J=6.9 Hz, 3H, CH₃) ppm. Due to the extreme toxicity of organomercury species, this material was discarded according to the Environmental Health and Safety regulations and procedures without obtaining further spectroscopic data.

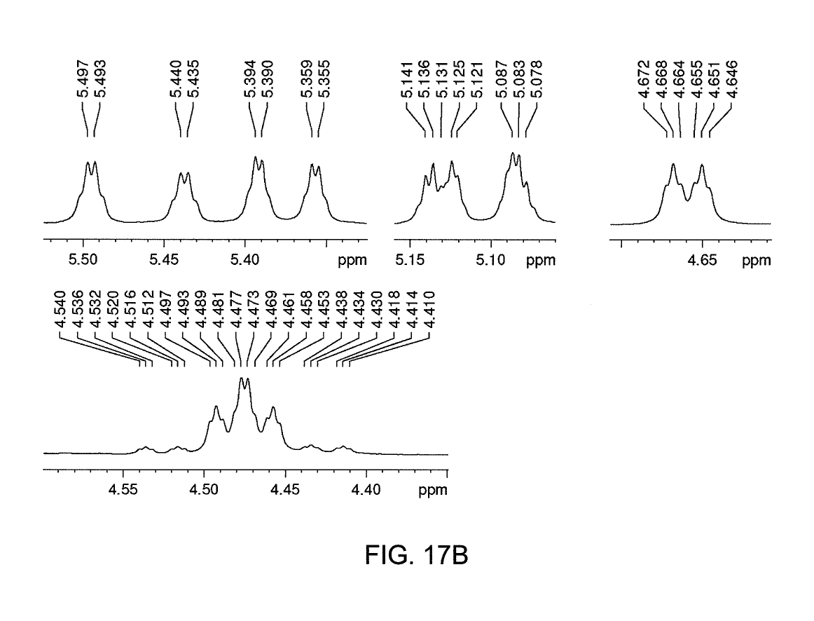

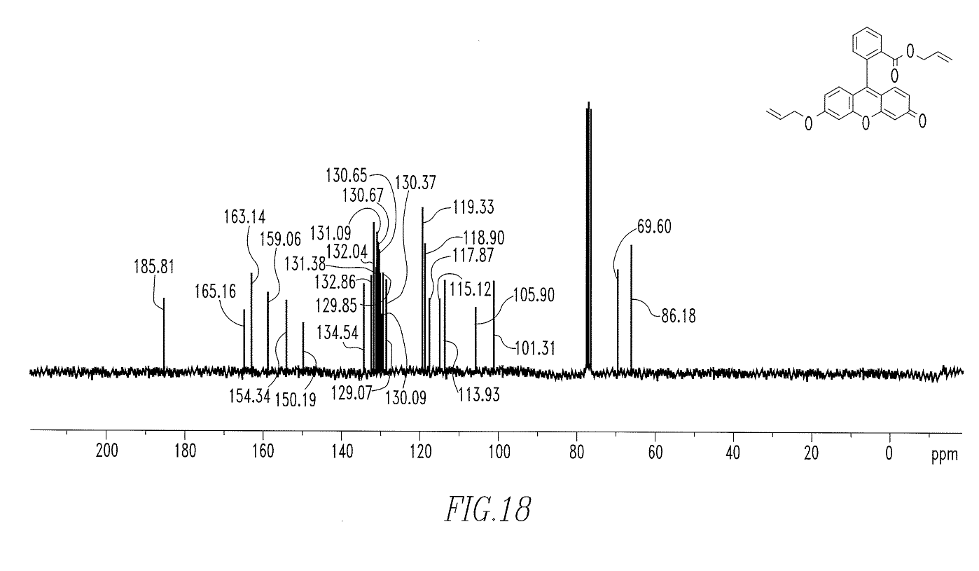

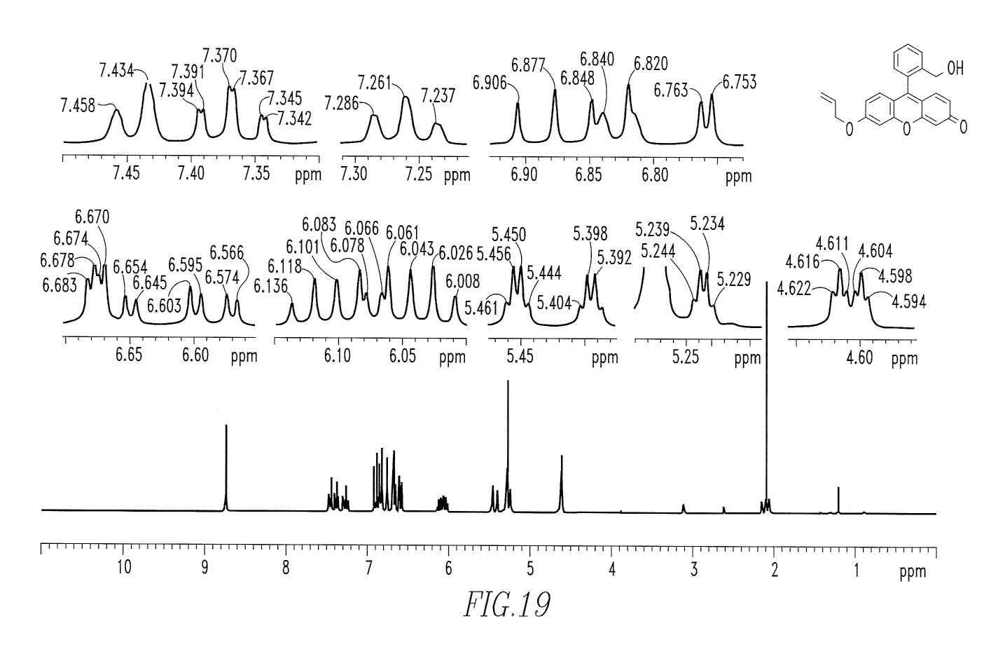

Compound 10 was prepared according to the following process. K₂CO₃ (4.15 g, 30.0 mmol) was added to a solution of fluorescein (3.32 g, 10.0 mmol) in DMF (20 mL) at 25° C. under a nitrogen atmosphere, followed by allyl bromide (2.60 mL, 30 mmol). After stirring for 48 h at 25° C., the reaction mixture was poured onto H₂O (500 mL). The resulting mixture was then extracted with EtOAc (3×200 mL), and the combined extracts were washed with H₂O (3×500 mL) and brine (500 mL), dried over Na₂SO₄, filtered, and evaporated under reduced pressure. The residue was recrystallized from hexanes-EtOAc to afford compound 10 (3.17 g, 77%) as an orange solid. Data for 10: m.p.=153-155° C.; R_{f}=0.34 (60% EtOAc in hexanes); IR (KBr pellet): νₘₐₓ=3054, 2986, 2932, 1727 (C═O), 1643 (C═O), 1595, 1517, 1481, 1256, 1211, 1106, 855, 759 cm⁻¹; ¹H NMR (300 MHz, CDCl₃, 293K, FIGS. 17a-b): δ=8.27 (dd, J=7.5, 1.5 Hz, 1H, Ar), 7.75 (ddd, J=7.5, 7.5, 1.5 Hz, 1H, Ar), 7.68 (ddd, J=7.5, 7.5, 1.5 Hz, 1H, Ar), 7.32 (dd, J=7.5, 1.5 Hz, 1H, Ar), 6.96 (d, J=2.4 Hz, 1H, Ar), 6.90 (d, J=9.0 Hz, 1H, Ar), 6.87 (d, J=9.6 Hz, 1H, Ar), 6.77 (dd, J=9.0, 2.4 Hz, 1H, Ar), 6.56 (dd, J=9.6, 1.8 Hz, 1H, Ar), 6.47 (d, J=1.8 Hz, 1H, Ar), 6.07 (dddd, J=17.4, 10.5, 5.4, 5.4 Hz, 1H, CH₂═CHCH₂OAr), 5.60 (dddd, J=17.4, 10.2, 6.0, 6.0 Hz, 1H, ArCO₂CH₂CH═CH₂), 5.46 (dd, J=17.4, 1.2 Hz, 1H, HₜᵣₐₙₛCH═CHCH₂OAr), 5.37 (dd, J=10.5, 1.2 Hz, 1H, HCH_{cis}═CHCH₂OAr), 5.14-5.08 (m, 2H, ArCO₂CH₂CH═CH₂), 4.66 (ddd, J=5.4, 1.2, 1.2 Hz, 2H, CH₂═CHCH₂OAr), 4.48 (dddd, J=12.9, 6.0, 1.2, 1.2 Hz, 1H, ArCO₂CHₐHCH═CH₂), 4.46 (dddd, J=12.9, 6.0, 1.2, 1.2 Hz, 1H, ArCO₂CHH_{b}CH═CH₂) ppm; ¹³C NMR (75 MHz, CDCl₃, 293K, FIG. 18): δ=185.8, 165.2, 163.1, 159.1, 154.3, 150.2, 134.5, 132.9, 132.0, 131.4, 131.1, 130.67, 130.65 130.4, 130.1, 129.9, 129.1, 119.3, 118.9, 117.9, 115.1, 113.9, 105.9, 101.3, 69.6, 66.2 ppm; HRMS (ESI+) m/z calcd. for C₂₆H₂₁O₅ [M]⁺ 413.1389, found 413.1373.

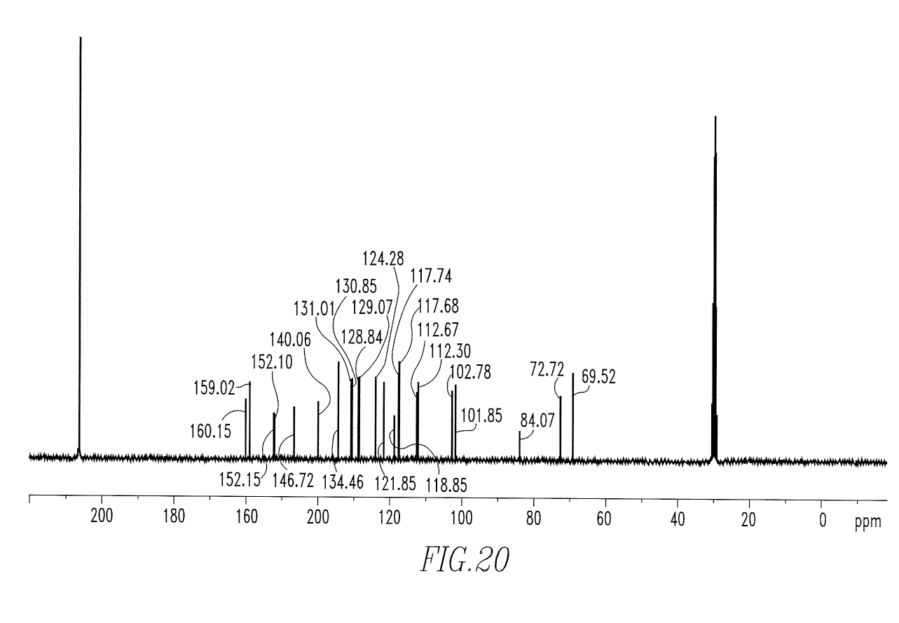

Compound 11 was prepared according to the following process. A solution of DIBALH (9.6 mL, 1.0 M in CH₂Cl₂) was added dropwise to a solution of compound 10 (0.83 g, 2.0 mmol) in CH₂Cl₂ (7.0 mL) over 15 min at −78° C. under a nitrogen atmosphere. The resulting solution was stirred at the same temperature for 10 min and then warmed to 25° C. After stirring at the same temperature for 2 h, Et₂O (5.0 mL) was added to the resulting solution at 0° C. with stirring, and then saturated aqueous NH₄Cl (3.5 mL) was added dropwise to the mixture at the same temperature. This mixture was warmed to 25° C. again and stirred for 1 h. The reaction mixture was then diluted with Et₂O (5.0 mL), and DDQ (0.45 g, 2.2 mmol) was added slowly to this mixture at 0° C. After being stirred for 1 h at 25° C., the mixture was filtered through a pad of Celite®, and the pad was rinsed with EtOAc. The filtrate was dried over Na₂SO₄, filtered, and evaporated under reduced pressure. The residue was purified by flash column chromatography (10 to 30% EtOAc in hexanes) on silica gel (180 mL) to afford compound 11 (701 mg, 98%) as a pale yellow solid. Data for compound 11: m.p.=144-146° C.; R_{f}=0.32 (30% EtOAc in hexanes); IR (KBr pellet): νₘₐₓ=3228 (broad, O—H), 2921, 2852, 1616 (C═O), 1501, 1517, 1456, 1430, 1335, 1224, 1180, 1003, 841, 759 cm⁻¹; ¹H NMR (300 MHz, acetone-d₆, 293K, FIG. 19): δ=7.44 (d, J=7.5 Hz, 1H, Ar), 7.37 (dd, J=7.5, 7.5 Hz, 1H, Ar), 7.26 (dd, J=7.5, 7.5 Hz, 1H, Ar), 6.89 (d, J=8.7 Hz, 1H, Ar), 6.84 (d, J=7.5 Hz, 1H, Ar), 6.83 (d, J=8.7 Hz, 1H, Ar), 6.76 (d, J=2.4 Hz, 1H, Ar), 6.67 (d, J=2.4 Hz, 1H, Ar), 6.66 (dd, J=8.7, 2.4 Hz, 1H, Ar), 6.58 (dd, J=8.7, 2.4 Hz, 1H, Ar), 6.07 (dddd, J=17.4, 10.5, 5.4, 5.4 Hz, 1H, CH₂═CHCH₂OAr), 5.42 (dd, J=17.4, 1.2 Hz, 1H, HₜᵣₐₙₛCH═CHCH₂OAr), 5.28 (s, 2H, ArCH₂OH), 5.24 (dd, J=10.5, 1.2 Hz, 1H, HCH_{cis}═CHCH₂OAr), 4.61 (ddd, J=5.4, 1.2, 1.2 Hz, 2H, CH₂═CHCH₂OAr) ppm; ¹³C NMR (75 MHz, acetone-d₆, 293K, FIG. 20): δ=160.2, 159.0, 152.2, 152.1, 146.7, 140.1, 134.5, 131.0, 130.9, 129.1, 128.8, 124.3, 121.9, 118.9, 117.74, 117.67, 112.7, 112.3, 107.8, 101.9, 84.1, 72.7, 69.5 ppm; HRMS (ESI+) m/z calcd. for C₂₃H₁₈O₄Na [M+Na]⁺ 381.1103, found 381.1072.

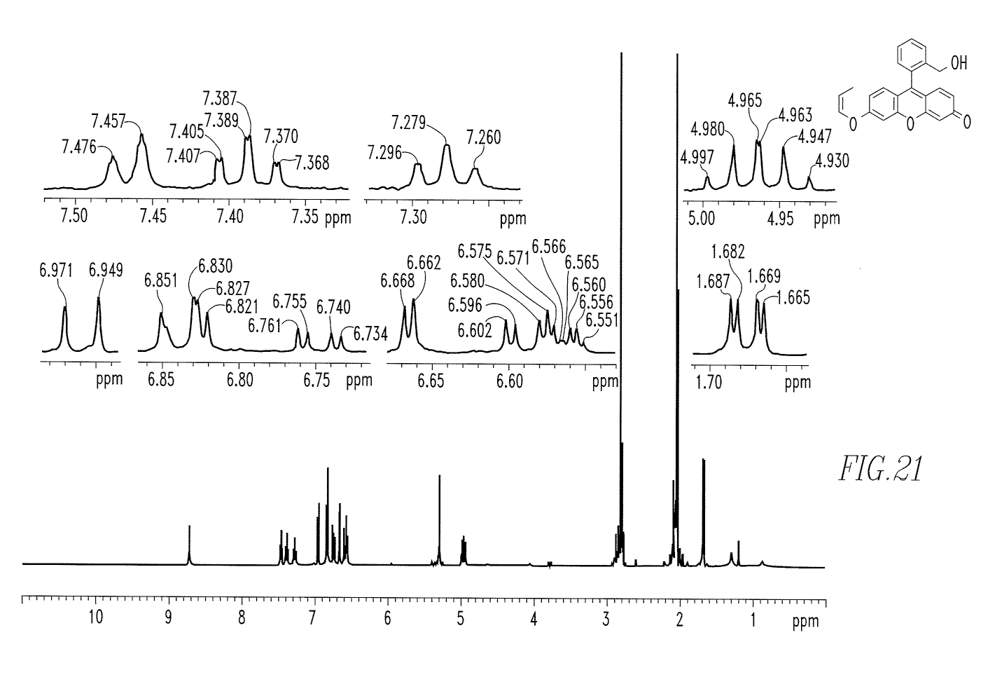

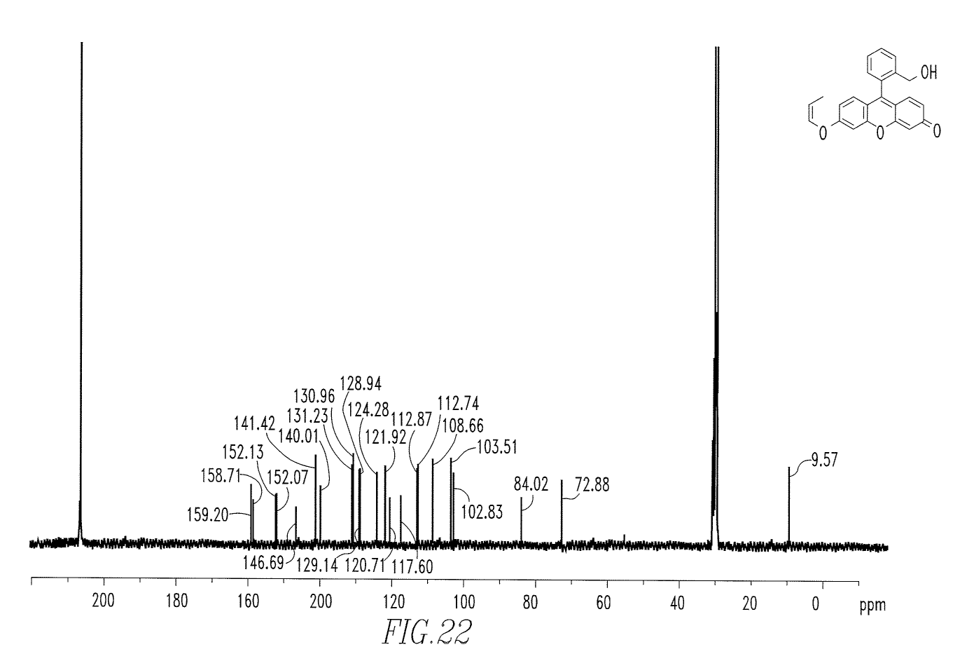

Compound 12 was prepared according to the following process. KOtBu (56 mg, 0.44 mmol) was added to a solution of compound 11 (72 mg, 0.20 mmol) in DMSO (1.7 mL) at 25° C. under a nitrogen atmosphere, and the resulting solution was heated in a 50° C. oil bath for 1 h. The reaction mixture was then poured onto ice-cold H₂O (10 mL), and the resulting mixture was extracted with EtOAc (2×10 mL). The combined organic layers were washed with brine (30 mL), dried over Na₂SO₄, filtered, and evaporated under reduced pressure. The residue was purified by flash column chromatography (10% to 30% EtOAc in hexanes) on silica gel (10 mL) to afford compound 12 (70 mg, 97%) as an orange solid. Data for 12: m.p.=96-98° C.; R_{f}=0.30 (40% EtOAc in hexanes); IR (KBr pellet): νₘₐₓ=3304 (broad, O—H), 3045, 2921, 2858, 1610 (C═O), 1497, 1459, 1426, 1270, 1177, 1109, 1023, 846, 804, 725 cm⁻¹; ¹H NMR (400 MHz, acetone-d₆, 293K, FIG. 21): δ=7.47 (d, J=7.6 Hz, 1H, Ar), 7.39 (dd, J=7.6, 7.6 Hz, 1H, Ar), 7.28 (dd, J=7.6, 7.6 Hz, 1H, Ar), 6.96 (d, J=8.8 Hz, 1H, Ar), 6.84 (d, J=8.8 Hz, 1H, Ar), 6.83 (d, 6.82 J=7.6 Hz, 1H, Ar), (d, J=2.4 Hz, 1H, Ar), 6.75 (dd, J=8.8 2.4 Hz, 1H, Ar), 6.67 (d, J=2.4 Hz, 1H, Ar), 6.60-6.55 (m, 2H, Ar and CH₃CH═CHOAr), 5.30 (s, 2H, ArCH₂OH), 4.96 (dq, J=6.8, 6.8 Hz, 1H, CH₃CH═CHOAr), 1.68 (dd, J=6.8, 1.6 Hz, 3H, CH₃CH═CHOAr) ppm; ¹³C NMR (100 MHz, acetone-d₆, 293K, FIG. 22): δ=159.2, 158.7, 152.12, 152.07, 146.7, 141.4, 140.0, 131.2, 131.0, 129.1, 128.9, 124.3, 121.9, 120.7, 117.6, 112.9, 112.7, 108.7, 103.5, 102.8, 84.0, 72.9, 9.6 ppm; HRMS (ESI+) m/z calcd. for C₂₃H₁₈O₄Na [M+Na]⁺ 381.1103, found 381.1083.

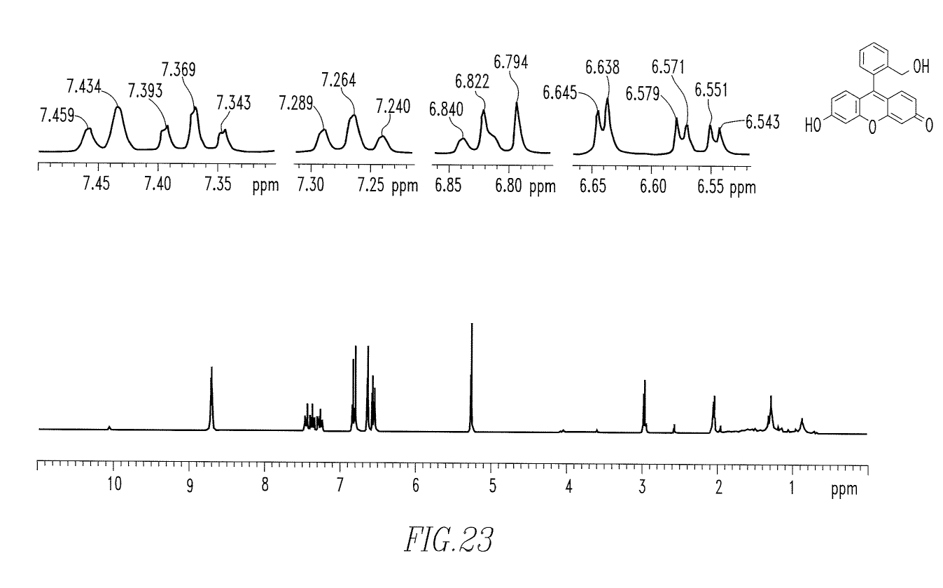

Compound 12 was converted to compound 13 according to the following process. HgCl₂ (18 mg, 67 μmol) was added to a solution of compound 12 (24 mg, 67 μmol) in a mixture of 1:9 DMSO/pH 4 buffer (26 mL). The resulting mixture was stirred at 25° C. for 1 h and extracted with EtOAc (2×20 mL). The combined organic layers were washed with brine (2×50 mL), dried over Na₂SO₄, filtered, and evaporated under reduced pressure. The residue was purified by flash column chromatography (10% to 70% EtOAc in hexanes) on silica gel (10 mL) to afford compound 13 (15 mg, 72%) as an orange solid. Data for 13: m.p.=>200° C.; R_{f}=0.32 (50% EtOAc in hexanes); IR (KBr pellet): νₘₐₓ=3430 (broad, O—H), 2869, 2600, 1602 (C═O), 1455, 1379, 1309, 1241, 1207, 1117, 860, 757 cm⁻¹; ¹H NMR (300 MHz, acetone-d₆, 293K, FIG. 23): δ=7.45 (d, J=7.5 Hz, 1H, Ar), 7.34 (dd, J=7.5, 7.5 Hz, 1H, Ar), 7.27 (dd, J=7.5 7.5 Hz, 1H, Ar), 6.83 (d, J=7.5 Hz, 1H, Ar) 6.81 (d, J=8.4 Hz, 2H, Ar), 6.64 (d, J=2.4 Hz, 2H, Ar), 6.56 (dd, J=8.4, 2.4 Hz, 2H, Ar), 5.26 (s, 2H, ArCH₂OH) ppm; ¹³C NMR (75 MHz, acetone-d₆, 293K, FIG. 24): δ=159.0, 152.3, 146.9, 140.2, 131.0, 129.1, 128.8, 124.4, 121.8, 117.9, 112.6, 102.8, 84.2, 72.6 ppm; HRMS (EI+) m/z calcd. for C₂₀H₁₅O₄ [M]⁺ 319.0970, found 319.0999.

Samples were prepared according to the following process. Mercury standard solution (20 ppm in 5% HNO₃) was purchased from the RICCA Chemical Company (Arlington, Tex.) and used as received. The other mercury standard solution (10,000 ppm in 2% HNO₃) was purchased from Ultra Scientific (North Kingstown, R.I.) and used as received. Omni Trace Ultra™ High Purity Acid HNO₃ (Hg<10 ppt) was purchased from EMD (Item number NX0408, Lot number 48157) and used as received. ARISTAR® ULTRA water was purchased from VWR (Catalog number 7732-18-5) and used as received. AgNO₃ was purchased from EMD and used as received. River water was collected from the Allegheny River on Jan. 29, 2008. The pH 3 buffer solution was made from a commercial pH 4 buffer (50 mM) and a pure HNO₃ solution. As discussed above, the test samples were also prepared by standard digestion methods to form a prepared test sample. Standard digestion methods include, but are not limited to, treating samples with a wet acid treatment, such as nitric acid at ambient temperature or above, or treating samples of oxidizing agents, such as Cl—Br, H₂O₂, and N-chlorosuccinimide (NCS) to transform various forms of organic and sulfur-bound (e.g., cysteine-bound) mercury species to sulfur-free inorganic mercury(II).

Metal solutions (1.0 mM): AuCl₃, BaCl₂, NiCl₂, CrCl₃, Pb(NO₃)₂, NaCl, MnCl₂.4H₂O, MgCl₂, CoCl₂, HgCl₂, AgNO₃, ZnCl₂, LiCl, CuCl₂.2H₂O, and CaCl₂ were dissolved in H₂O. FeCl₃, CdCl₂.2.5H₂O, KCl, Rh(PPh₃)₃, and RuCl₃ were dissolved in MeOH. PtCl₂ was dissolved in MeOH/acetone (1:1). Pd standard solution (High-Purity Standards, Cat. No. 100038-1) was diluted with 1% HNO₃. The resulting solution was used as the Pd solution.

Fluorescence measurements were taken according to the following process. All samples were incubated at 25° C., and the pH values of the solutions were adjusted to an appropriate pH range (pH>5 for 1, pH>8 for 13) by the addition of 1.23 M phosphate pH 7 buffer (for 1, 4.0% of the total volume of a reaction solution) or a 1:5 mixture of 1.23 M phosphate pH 7 buffer and 500 mM borate pH 10 buffer (for 13, 24% of the total volume of reaction solution). The resulting samples were vortexed for 5 s prior to fluorescence measurement. Fluorescence spectra were recorded in a 1×1-cm disposable cuvette (VWR; catalog number 58017-880) on a Jobin Yvon Fluor® Max-3 spectrometer under the control of a Windows-based PC running FluorEssence software. The samples were excited at 497 nm and the emission intensities were collected at 523 nm (for phenol 1) or 515 nm (for compound 13). All spectra were corrected for emission intensity using the manufacturer-supplied photomultiplier curves.

The pH dependence of Hg detection is shown in FIGS. 1A and 5A. Reaction conditions: [Hg(II)]=0.30 μM, [vinyl ether 3 or 12]=1.0 μM, 0.05% DMSO in buffer (5.0 mL), 25° C., 1 h. Protocol: A 100 μM Hg standard solution (15 μL each) was added to a pH 3, 4, 5, 6, or 7 buffer (4.98 mL each). A 1.0 mM solution of vinyl ether 3 or 12 in 1:1 DMSO/50 mM phosphate pH 8 buffer (5.0 μL) was added to each of these solutions, and the resulting samples were shaken for 3 s and incubated at 25° C. for 1 h before fluorescence measurement. Note: For the pH 3 sample, an additional 1.0 mL of 500 mM borate pH 10 buffer was required to obtain a pH>5 solution.

Metal selectivity is shown in FIGS. 1B and 5B). Reaction conditions: [metal]=5.0 μM, [vinyl ether 3 or 12]=1.0 μM, 0.05% DMSO in a 50 mM phthalate pH 4 buffer (5.0 mL), 25° C., 1 h. Protocol: Each of 1.0 mM solutions of metal reagents in appropriate solvents (25 μL) was added to a 50 mM phthalate pH 4 buffer (4.97 mL). A 1.0 mM solution of vinyl ether 3 or 12 in 1:1 DMSO/50 mM phosphate pH 8 buffer (5.0 μL) was added to these solutions, and the resulting samples were shaken for 3 s and incubated at 25° C. for 1 h before fluorescence measurement.

Time-course of the oxymercuration reaction is shown in FIGS. 1C, 6A, 6B, and 6C. Reaction conditions: [Hg(II)]=0.30 μM, [vinyl ether 3, 12, or 14]=1.0 μM, 0.05% DMSO in a 50 mM phthalate pH 4 buffer or a 50 mM pH 7 phosphate buffer (20 mL), 25° C. Protocol: A 100 μM Hg standard solution (60 μL) was added to a 50 mM phthalate pH 4 buffer (FIGS. 1C, 6A and 6B) or a 50 mM phosphate pH 7 buffer (FIG. 6C) (20 mL). A 1.0 mM solution of vinyl ether 3, 12, or 14 in 1:1 DMSO/50 mM phosphate pH 8 buffer (20 μL) was added to each of the solutions, and the resulting reaction solutions were shaken for 3 s and incubated at 25° C. before fluorescence measurement. A fraction (1.5 mL) of each of these reaction solutions was taken for the measurement at 10, 20, 30, 40, 50, 60, 90, 120, 240, 480, and 1140 min. For FIG. 6B, this measurement was carried out every 1 min for 18 min with 1.0 mL of the reaction solution.

Titration curves are shown in FIGS. 1D and 5D. Reaction conditions: [Hg(II)]=0, 1, 2, 4, 8, 16, and 32 ppb, [NCS]=10 μM for NCS(+) samples and 0 μM for NCS(−) samples, [vinyl ether 3 or 12]=1.0 μM, 0.05% DMSO in a 50 mM phthalate pH 4 buffer (5.0 mL), 25° C., 1 h. Protocol: A 0, 200, 400, 800, 1600, 3200, or 6400 ppb solution of HgCl₂ in 5% HNO₃ (25 μL) and a 2.0 mM solution of NCS in water (25 μL) were added to each of 50 mM phthalate pH 4 buffers (4.95 mL). A 1.0 mM solution of vinyl ether 3 or 12 in 1:1 DMSO/50 mM phosphate pH 8 buffer (5.0 μL) was added to each of these solutions, and the resulting solutions were shaken for 3 s and incubated at 25° C. for 1 h before fluorescence measurement.

Hg detection in the presence of other metal is shown in FIG. 2A (white bar). Reaction conditions: [Hg(II)]=2.5 μM, [other metal]=25 M, [vinyl ether 3]=1.0 M, 0.05% DMSO in a 50 mM phthalate pH 4 buffer (5.0 mL), 25° C., 1 h. Protocol: A 100 μM Hg standard solution in 5% HNO₃ (125 μL) and each of the 1.0 mM metal solutions (125 μL) were added to each of 50 mM phthalate pH 4 buffer (4.75 mL). Each of these solutions was treated with a 1.0 mM solution of vinyl ether 3 in 1:1 DMSO/50 mM phosphate pH 8 buffer (5.0 μL). The resulting solutions were shaken for 3 s and incubated at 25° C. for 1 h before fluorescence measurement.

The effect of chloride or nitrate ion is shown in FIGS. 2B and 13. Reaction conditions: [Hg(II)]=2.5 μM, [Cl⁻ or NO₃⁻]=1.0 mM, [vinyl ether 3 or 12]=1.0 μM, 0.05% DMSO in a 50 mM phthalate pH 4 buffer (5.0 mL), 25° C., 1 h. Protocol: A 1.0 mM HgCl₂ solution in 5% HNO₃ (12.5 μL) and a 100 mM NaCl or NaNO₃ solution in water (50 μL) were added to a 50 mM phthalate pH 4 buffer (4.93 mL). Each of these solutions was treated with a 1.0 mM solution of vinyl ether 3 or 12 in 1:1 DMSO/50 mM phosphate pH 8 buffer (5.0 μL). The resulting samples were shaken for 3 s and incubated at 25° C. for 15 min (FIG. 2B) or 1 h (FIG. 5C) before fluorescence measurement.

The interference of inorganic materials with AgNO₃ is shown in FIGS. 2A (gray bar) and 5c. Reaction conditions for FIG. 2A: [Hg(II)]=2.5 μM, [other metal]=25 μM, [AgNO₃]=100 μM, [vinyl ether 3]=1.0 μM, 0.05% DMSO in a 50 mM phthalate pH 4 buffer (5.0 mL), 25° C., 1 h. Protocol: A 1.0 mM HgCl₂ solution in 5% HNO₃ (12.5 μL) and a 1.0 mM solution of another metal (125 μL) were added to a 50 mM phthalate pH 4 buffer (4.86 mL). These solutions were vortexed for 5 s, and then a 10 mM solution of AgNO₃ in ultra pure water (50 μL) was added to the resulting solutions. After vortexing for 5 s, a 1.0 mM solution of vinyl ether 3 in 1:1 DMSO/50 mM phosphate pH 8 buffer (5.0 μL) was added to these solutions, and the resulting samples were shaken for 3 s and incubated at 25° C. for 1 h before fluorescence measurement.

Reaction conditions for FIG. 5C are as follows: [Hg(II)]=1.0 M, [other metal]=10 M, [AgNO₃]=2.0 mM, [compound 12]=1.0 μM, 0.05% DMSO in a 50 mM phthalate pH 4 buffer (5.0 mL), 25° C., 1 h. Protocol: A 1.0 mM HgCl₂ solution in 5% HNO₃ (5.0 μL) and a 1.0 mM solution of another metal (50 μL) were added to a 50 mM phthalate pH 4 buffer (4.84 mL). These solutions were vortexed for 5 s, and then a 100 mM solution of AgNO₃ in ultra pure water (100 μL) was added to the resulting solutions. After vortexing for 5 s, a 1.0 mM solution of compound 12 in 1:1 DMSO/50 mM phosphate pH 8 buffer (5.0 μL) was added to these solutions, and the resulting samples were shaken for 3 s and incubated at 25° C. for 1 h before fluorescence measurement.

Detection of Hg in river water by probe vinyl ether 3 is shown in FIGS. 3A and 3B. Reaction conditions for FIG. 3A: [Hg(II)]=440 ppb (550 ppb=2.75 μM in river before dilution), [AgNO₃]=2.0 mM, [vinyl ether 3]=1.0 μM, 0.05% DMSO in a 50 mM phthalate pH 4 buffer (5.09 mL), 25° C., 1 h. Protocol: River water (3.98 mL) was spiked with a 440 μM solution of HgCl₂ in 5% HNO₃ (25 μL). This solution was treated with a 250 mM phthalate pH 4 buffer (1.0 mL) and then a 100 mM solution of AgNO₃ in ultra pure water (100 μL). After vortexing for 5 s, a 1.0 mM solution of vinyl ether 3 in 1:1 DMSO/50 mM phosphate pH 8 buffer (5.0 μL) was added to this solution, and the resulting sample was shaken for 3 s and incubated at 25° C. for 1 h before fluorescence measurement.

Reaction conditions for FIG. 3B are as follows: [Hg(II)]=0, 1, 2, 4, 8, 16, 32, 64, 128, and 256 ppb, [AgNO₃]=2.0 mM, [vinyl ether 3]=1.0 μM, 0.05% DMSO in a 50 mM phthalate pH 4 buffer (5.1 mL), 25° C., 1 h. Protocol: A 0, 0.2, 0.4, 0.8, 1.6, 3.2, 6.4, 12.8, 25.6, or 51.2 ppm solution of HgCl₂ in 5% HNO₃ (25 μL) was spiked into each of river water samples (4.0 mL each). Each of these solutions was treated with a 250 mM phthalate pH 4 buffer (1.0 mL each) and then a 100 mM solution of AgNO₃ in ultra pure water (100 μl, each). After being shaken for 3 s, a 1.0 mM solution of vinyl ether 3 in 1:1 DMSO/50 mM phosphate pH 8 buffer (5.0 μL) was added to these solutions, and the resulting samples were shaken for 3 s and incubated at 25° C. for 1 h before fluorescence measurement.

Hg detection in the presence of organic compound is shown in FIG. 3C). Reaction conditions: ([Hg(II)]=16 ppb, [organic contaminant]=80 ppb, [AgNO₃]=2 mM, [vinyl ether 3]=1 μM, 1.3% DMSO in a 50 mM phthalate pH 4 buffer (1.30 mL), 25° C., 1 h. Protocol: River water (1.0 mL) was spiked with a 10 μM HgCl₂ solution in 5% HNO₃ (10 μL) and a 10 ppm solution of an organic contaminant in DMSO (phenol, acetophenone, caffeine, or cholesterol, 10 μL). Each of these solutions contained 0.1 μM (20 ppb) Hg(II) and 100 ppb compound. Each of the resulting solutions was treated with a 250 mM phthalate pH 4 buffer (250 μl each) and then a 100 mM solution of AgNO₃ in ultra pure water (25 μL). After vortexing for 5 s, a 0.1 mM solution of vinyl ether 3 in 1:1 DMSO/50 mM phosphate pH 8 buffer (12.5 μL) was added to this solution, and the resulting sample was shaken for 3 s and incubated at 25° C. for 1 h before fluorescence measurement.

Detection of Hg from dental amalgam is shown in FIG. 3D. Reaction conditions: Two teeth filled with mercury amalgam, [cysteine]=50 μM, [NCS]=500 μM, [vinyl ether 3]=1.0 μM, 0.05% DMSO in a 50 mM phthalate pH 4 buffer (4.06 mL), 25° C., 1 h. Protocol: Two teeth filled with an amalgam were added to a 20 mM solution of cysteine in water (2.0 mL), and the resulting mixture was shaken (200 rpm) for 1 h at 37° C. A portion (5.0 μL) of the resulting solution was added to a 50 mM phthalate pH 4 buffer (2.0 mL). After being shaken for 3 s, a 20 mM solution of NCS in water (50 μL) and a 1.0 mM solution of vinyl ether 3 in 1:1 DMSO/50 mM phosphate pH 8 buffer (2.0 μL) were added to the resulting solution. A negative control sample was prepared by adding two teeth filled with an amalgam into water (2.0 mL) and shaken (200 rpm) for 1 h at 37° C. A portion (5.0 μL) of the resulting mixture was added to a 50 mM phthalate pH 4 buffer (2.0 mL). This mixture was then treated with a 20 mM solution of NCS in water (50 μL) and a 1.0 mM solution of vinyl ether 3 in 1:1 DMSO/50 mM phosphate pH 8 buffer (2.0 μL). The resulting samples were shaken for 3 s and incubated at 25° C. for 1 h before fluorescence measurement.

The pH titration of compound 13 is shown in FIG. 4A. An aqueous solution (100 mL) containing compound 13 (5 μM) and NaCl (1.0 M) was prepared. To one half of this mixture solution (50 mL) was added a small magnetic stir bar in a vial with a pH electrode. The pH of the solution was changed by adding 0.1 N or 1 N HCl solution dropwise while stirring and the fluorescence spectrum was recorded at −0.3 pH intervals. The other half of this mixture solution (50 mL) was titrated with 0.1 N or 1 N NaOH solution. In order to calculate the pKa, the pH dependence of fluorescence spectra was analyzed using the following equation: pH=pKa−log [(Fₘₐₓ−F)/(F−Fₘᵢₙ)].

The effect of anion is shown in FIG. 5E. Reaction conditions: [Hg(II)]=0.5 M, [Cl⁻, Br⁻, I⁻, SO₄²⁻, or NO₃⁻]=2.0 mM, [compound 12]=1.0 μM, 0.05% DMSO in a 50 mM phthalate pH 4 buffer (5.0 mL), 25° C., 1 h. Protocol: A 0.5 mM HgCl₂ solution in 5% HNO₃ (5.0 μL) and a 100 mM solution of either NaCl, NaBr, NaI, Na₂SO₄, or NaNO₃ in water (100 μL) were added to a 50 mM phthalate pH 4 buffer (4.90 mL). A 1.0 mM solution of compound 12 in 1:1 DMSO/50 mM phosphate pH 8 buffer (5.0 μL) was added to these solutions, and the resulting samples were shaken for 3 s and incubated at 25° C. for 1 h before fluorescence measurement.

Detection of Hg spiked in river water by probe compound 12 is shown in FIG. 7. Reaction conditions: [Hg(II)]=0, 0.25, 0.5, 1, 2, and 4 ppb, [AgNO₃]=2.0 mM, [compound 12]=1.0 μM, 0.5% DMSO in a 50 mM phthalate pH 4 buffer (1.02 mL), 25° C., 1 h. Protocol: A 50 mM phthalate pH 4 buffer (20 mL) was dried under reduced pressure to obtain a solid in a test tube. River water (20 mL) was passed through a syringe filter and poured into the test tube to adjust the pH to 4. A 0, 62.5, 125, 250, 500, or 1000 ppb solution of HgCl₂ in 5% HNO₃ (4 μL) was spiked into each of the resulting river water samples (1.0 mL each). A 100 mM solution of AgNO₃ in ultra pure water (20 μL each) was added to each of these samples, and the resulting mixtures were vortexed for 5 s and centrifuged (1 min, 2000 rpm) to remove precipitates. A 0.1 mM solution of compound 12 in 1:1 DMSO/50 mM phosphate pH 8 buffer (10 μL) was added to each of these solutions, and the resulting samples were shaken for 3 s and incubated at 25° C. for 1 h before fluorescence measurement.

To measure interference of oxymercuration by chloride ions, a solution of vinyl ether 3 (8.5 mg, 20 μmol) in DMSO (200 μL) was treated with pH 4.0 buffer (50 mM, 3.8 mL), NaCl (0.58 g, 10 mmol), and then HgCl₂ (14 mg, 50 μmol) at 25° C. After stirring for 1 h at 25° C., the reaction mixture was extracted with EtOAc (3×5 mL). The combined organic layers were washed with H₂O (2×15 mL) and brine (15 mL), dried over Na₂SO₄, filtered, and concentrated under reduced pressure. The crude mixture was dissolved in acetone-d₆ and analyzed by ¹H NMR spectroscopy (see FIG. 25).

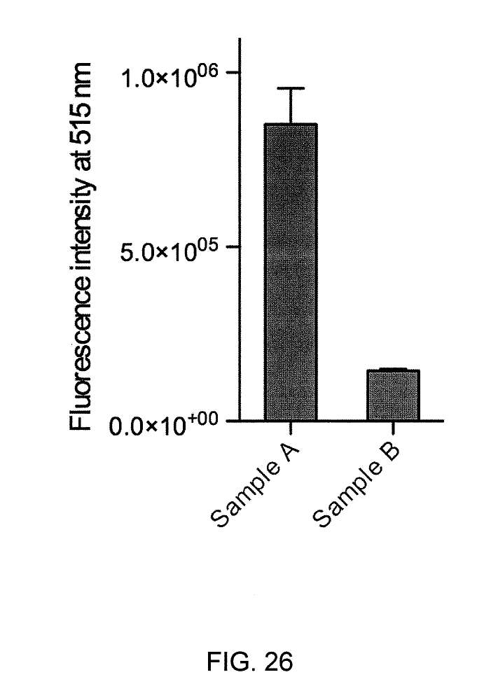

One embodiment of the present invention detects mercury in wastewater using the following process. A pretreatment of 10 N NaOH (500 μL) was added to samples A and B from a coal-fired power plant (10 mL). This operation was important to remove the fluorescence contaminants, if any. The precipitated solid was removed by centrifuge (4000 rpm, 15 min). The supernatant was neutralized with 69% OmniTrace Ultra™ High Purity Acid HNO₃ (Hg<10 ppt), and 5.0 mL of the resulting solution was added to the salt of pH 4 buffer (dried salts from 5.0 mL of 50 mM phthalate buffer). For detection, a solution of AgNO₃ (20 μL, 100 mM in ARISTAR® ULTRA water, [AgNO₃]_{final}=2.0 mM) was added to the treated samples A and B (1.0 mL, triplicate), and the resulting mixtures were shaken by vortex for 2 s. A solution of compound 12 (10 μL, 0.1 mM in 50% DMSO/50 mM phosphate pH 8 buffer, [probe]_{final}=1.0 μM) was added to the resulting mixtures. After incubating at 25° C. for 1 h, the reaction mixtures were basified with 500 mM borate pH 10 buffer (200 μL) and 1.23 M phosphate pH 7 buffer (40 μL). The solid precipitates were removed by centrifuge (2000 rpm, 1 min), and the resulting supernatants were used for the fluorescence measurement. The signal from Sample A (see FIG. 26) indicated that ˜8 ppb Hg was present in the original sample. No increase in signal was from Sample B, indicating that there was not a detectable amount of Hg in this sample. The ICP-MS analysis of samples A and B indicated that the Hg concentrations in these sampled were 2 ppb and below 500 ppt, respectively.

While the disclosure has been described in detail and with reference to specific embodiments thereof, it will be apparent to one skilled in the art that various changes and modifications can be made therein without departing from the spirit and scope of the embodiments. Thus, it is intended that the present disclosure cover the modifications and variations of this disclosure provided they come within the scope of the appended claims and their equivalents

What is claimed is:

1. A method to detect mercury(II) ions in a sample, the method comprising steps of:
preparing the sample by digestion methods to transform organic and sulfur-bound mercury species to sulfur-free inorganic mercury(II);
performing an oxymercuration reaction with the sample in the presence of about 0.01 μM to about 100 μM of a compound having a 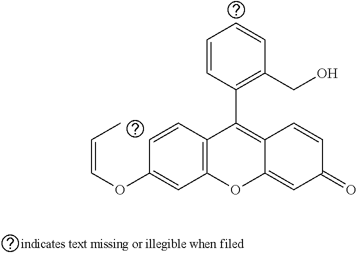
structure and about 0 mM to 100 mM of AgNO₃ in about 0% to about 5% DMSO in pH about 3 to about 7;
adjusting a temperature of the oxymercuration reaction between about 0° C. to about 100° C. and holding the temperature for about 0.1 hour to about 2 hours to create a resulting solution;
basifying the resulting solution to adjust the pH to between about 7 to about 11 to form a basified solution; and
measuring the fluorescence signals of the resulting solution.

2. The method according to claim 1, wherein the compound structure further comprises: 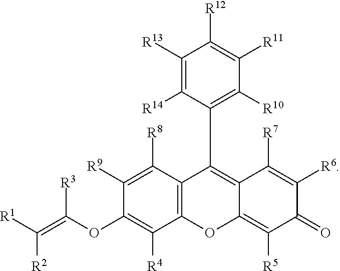
where:
R¹═H, alkyl, aryl, or other functional groups,
R²═H, alkyl, aryl, or other functional groups,
R³═H, alkyl, aryl, or other functional groups,
R⁴═H, alkyl, aryl, or other functional groups,
R⁵═H, alkyl, aryl, or other functional groups,
R⁶═H, alkyl, aryl, or other functional groups, but not Cl or F,
R⁷ and R⁸═H, alkyl, aryl, F, Cl, Br, aryl, or other functional groups,
R⁹═H, alkyl, aryl, or other functional groups, but not Cl or F,
R¹⁰ and R¹⁴═H, hydroxymethyl, alkyl, aryl, or other functional groups, excluding an electron-withdrawing group such as carboxylate, formyl, and ketone,
R¹¹ and R¹³═H, alkyl, aryl, F, Cl, Br, aryl, or other functional groups,
R¹²═H, alkyl, aryl, or other functional groups, excluding an electron-withdrawing group, wherein the electron-withdrawing group is selected from a group consisting of carboxylate, formyl, and ketone.

3. The method according to claim 2, wherein the electron-withdrawing group is selected from a group consisting of carboxylate, formyl, and ketone.

4. The method according to claim 1, wherein the step of performing the oxymercuration reaction further comprises about 1 μM to about 75 μM of the compound.

5. The method according to claim 1, wherein the step of performing the oxymercuration reaction further comprises about 10 μM to 50 μM of the compound.

6. The method according to claim 1, wherein the step of performing the oxymercuration reaction further comprises about 20 μM to about 40 μM of the compound.

7. The method according to claim 1, wherein the step of performing the oxymercuration reaction further comprises about 25 μM to about 35 μM of the compound.

8. The method according to claim 1, wherein the step of performing the oxymercuration reaction further comprises about 1 μM of the compound.

9. The method according to claim 1, wherein the step of performing the oxymercuration reaction further comprises about 67 μM of the compound.

10. The method according to claim 1, wherein the step of performing the oxymercuration reaction further comprises about 0.1 mM to about 90 mM of the AgNO₃.

11. The method according to claim 1, wherein the step of performing the oxymercuration reaction further comprises about 10 mM to about 80 mM of the AgNO₃.

12. The method according to claim 1, wherein the step of performing the oxymercuration reaction further comprises about 20 mM to about 60 mM of the AgNO₃.

13. The method according to claim 1, wherein the step of performing the oxymercuration reaction further comprises about 30 mM to about 50 mM of the AgNO₃.

14. The method according to claim 1, wherein the step of adjusting the temperature of the oxymercuration reaction further comprises adjusting the temperature between about 0° C. to about 25° C.

15. The method according to claim 1, wherein the step of adjusting the temperature of the oxymercuration reaction further comprises adjusting the temperature between about 0° C. to about 85° C.

16. The method according to claim 1, wherein the step of adjusting the temperature of the oxymercuration reaction further comprises adjusting the temperature between about 10° C. to about 40° C.

17. The method according to claim 1, wherein the step of adjusting the temperature of the oxymercuration reaction further comprises adjusting the temperature between about 20° C. to about 30° C.

18. The method according to claim 1, wherein the step of adjusting the temperature further comprises the step of holding the temperature for about 0.25 hours to 1.5 hours.

19. The method according to claim 1, wherein the step of adjusting the temperature further comprises the step of holding the temperature for about 0.5 hours to 1.0 hours.

20. The method according to claim 1, wherein the step of adjusting the temperature further comprises the step of holding the temperature for about 0.7 hours to 0.8 hours.

21. The method according to claim 1, wherein the step of adjusting the temperature of the oxymercuration reaction occurs at about 25° C. and holding the temperature for about 1 hour to create the resulting solution.

22. The method according to claim 1, wherein the step of adjusting the temperature of the oxymercuration reaction occurs at about 25° C. and holding the temperature for about 15 minutes to create the oxymercuration reaction to create the resulting solution.

23. A fluorogenic chemodosimeter comprising a structure of:

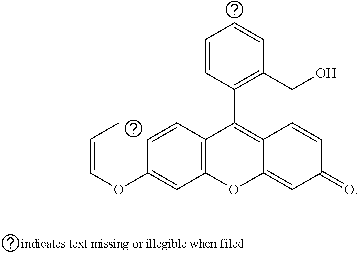

24. The fluorogenic chemodosimeter according to claim 23, wherein the compound structure further comprises: 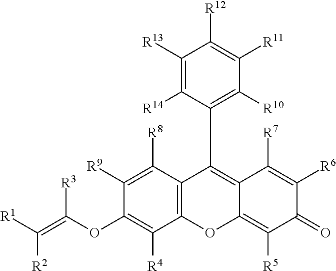
where:
R¹═H, alkyl, aryl, or other functional groups,
R²═H, alkyl, aryl, or other functional groups,
R³═H, alkyl, aryl, or other functional groups,
R⁴═H, alkyl, aryl, or other functional groups,
R⁵═H, alkyl, aryl, or other functional groups,
R⁶═H, alkyl, aryl, or other functional groups, but not Cl or F,
R⁷ and R⁸═H, alkyl, aryl, F, Cl, Br, aryl, or other functional groups,
R⁹═H, alkyl, aryl, or other functional groups, but not Cl or F,
R¹⁰ and R¹⁴═H, hydroxymethyl, alkyl, aryl, or other functional groups, excluding an electron-withdrawing group such as carboxylate, formyl, and ketone,
R¹¹ and R¹³═H, alkyl, aryl, F, Cl, Br, aryl, or other functional groups,
R¹²═H, alkyl, aryl, or other functional groups, excluding an electron-withdrawing group, wherein the electron-withdrawing group is selected from a group consisting of carboxylate, formyl, and ketone.

25. The fluorogenic chemodosimeter according to claim 24, wherein the electron-withdrawing group is selected from a group consisting of carboxylate, formyl, and ketone.
